(19) United States
(12) Patent Application Publication
(10) Pub. No.: US 2025/0241962 A1
(43) Pub. Date: Jul. 31, 2025

(54) METHODS AND COMPOSITIONS FOR INCREASING NEURONAL ACTIVITY AND SYNAPTIC PLASTICITY

(71) Applicant: SanBio, Inc., Oakland, CA (US)

(72) Inventor: Ricardo Luiz AZEVEDO-PEREIRA, San Mateo, CA (US)

(73) Assignee: SanBio, Inc., Oakland, CA (US)

(21) Appl. No.: 19/039,149

(22) Filed: Jan. 28, 2025

Related U.S. Application Data
(60) Provisional application No. 63/626,785, filed on Jan. 30, 2024.

(51) Int. Cl. A61K 35/28; C12N 5/0775

(52) U.S. Cl. CPC A61K 35/28; C12N 5/0663; C12N 2501/42; C12N 2510/00

(57) ABSTRACT

Disclosed are compositions and methods for inducing tonic release of glutamate and increasing synapse formation. For example, disclosed is a method for inducing tonic release of glutamate comprising administering vandefitemcel to a region of the brain of a subject. The vandefitemcel can be cells descended from mesenchymal stem cells (MSCs) transiently-transfected by a polynucleotide encoding a Notch intracellular domain (NICD). Also, for example, disclosed is a method of increasing synapse formation in neurons. The method can comprise capturing functional magnetic resonance imaging (fMRI) scans of regions of the brain of the subject, selecting a region of the brain showing neuronal activity based on the fMRI scans, and administering vandefitemcel to the region of the brain selected comprising the neurons.

### CROSS-REFERENCE TO RELATED APPLICATION

This application claims the benefit of U.S. Provisional Application No. 63/626,785 filed on Jan. 30, 2024, the content of which is incorporated herein by reference in its entirety.

### TECHNICAL FIELD

The present disclosure relates generally to the field of cell therapies and, more specifically, to methods and compositions for increasing neuronal activity and synaptic plasticity in a subject in need thereof.

### BACKGROUND

Over the past several decades, mesenchymal stem cell-based (MSC-based) therapies have emerged as a new strategy for treating neurological disorders and injuries such as stroke, traumatic brain injury (TBI), amyotrophic lateral sclerosis (ALS), and spinal cord injury (SCI) [1, 2]. These neurological disorders or injuries often involve the impairment of certain cognitive functions due to disruptions in how neurons communicate. In order to re-establish neuronal communication and promote functional recovery, there must be an increase in neuronal activity and synaptic plasticity that allows for the repair of certain neural pathways or the creation of new neural pathways.

MSCs have low immunogenicity and can be isolated from different adult and birth tissues and cultured at great expansion capacity [3]. Vandefitemcel, also known as SB623 cells, are a type of human bone marrow-derived MSC cells. Recent studies have shown that intracerebral implantation of vandefitemcel or SB623 cells was safe and could improve a patient's motor functions [4, 5]. However, little is known about the effect of MSCs on the electrophysiology of neurons (and, especially, human neurons) after their implantation or transplantation.

While neurons for research can come from a variety of sources, one renewable source of human neurons that do not require invasive biopsies of human central nervous system (CNS) tissue is human induced pluripotent stem cells (iPSCs). Human iPSCs can be differentiated into a range of somatic cells including types of neurons such as glutamatergic and GABAergic neurons [6].

Moreover, multielectrode arrays (MEAs) have been used in the past to measure the electrophysiology of neurons [7]. MEAs typically include a special multi-well culture plate with small electrodes embedded at the bottom of each well. Extracellular spontaneous action potentials are recorded from neuronal cells cultured directly on top of the recording electrodes.

Therefore, there is a need for safe and effective cell-based therapies for increasing neuronal activity and synaptic plasticity. Such therapies should be capable of being tested on human iPSC-derived neurons using state-of-the-art MEA measurement systems.

### SUMMARY

In some aspects, disclosed is a method for inducing, triggering, or causing tonic release of glutamate in a subject. The method comprises administering vandefitemcel to a region of the brain of the subject. The vandefitemcel can be cells descended from mesenchymal stem cells (MSCs) transiently-transfected by a polynucleotide encoding a Notch intracellular domain (NICD).

In some aspects, administering vandefitemcel to a region of the brain of the subject further comprises administering the vandefitemcel by intracerebral implantation.

In some aspects, the vandefitemcel can tonically release glutamate to neurons of the brain when implanted via intracerebral implantation.

In some aspects, administering vandefitemcel to a region of the brain of the subject further comprises injecting the vandefitemcel at multiple sites within the region of the brain.

In some aspects, administering vandefitemcel to a region of the brain of the subject further comprises administering the vandefitemcel stereotactically via a burr hole in the skull of the subject.

In some aspects, the region of the brain is the forebrain of the subject including at least one of the cerebrum, the thalamus, and the hypothalamus of the subject.

In some aspects, the region of the brain can be a site of injury or disease.

In some aspects, the region of the brain can be the hippocampus of the subject.

In some aspects, administering vandefitemcel to a region of the brain of the subject further comprises administering the vandefitemcel by parenteral administration.

In some aspects, the vandefitemcel can be suspended in a sterile isotonic crystalloid solution.

In some aspects, administering vandefitemcel to a region of the brain of the subject further comprises administering between about 1.0 million cells and 10.0 million cells.

In some aspects, the vandefitemcel can be made by a method comprising providing a culture of MSCs; contacting the culture of MSCs with the polynucleotide encoding the NICD, wherein the polynucleotide does not encode a full-length Notch protein; selecting cells that comprise the polynucleotide; and further culturing the selected cells in the absence of selection for the polynucleotide.

In some aspects, the MSCs can be human bone marrow-derived cells.

In some aspects, the method can further comprise capturing functional magnetic resonance imaging (fMRI) scans of regions of the brain of the subject, selecting one region of the brain showing neuronal activity based on the fMRI scans, and administering the vandefitemcel to the one region of the brain.

In some aspects, disclosed is a method for increasing synapse formation in neurons. The method can comprise administering vandefitemcel to a region of the brain of the subject comprising the neurons. The vandefitemcel can be cells descended from mesenchymal stem cells (MSCs) transiently-transfected by a polynucleotide encoding a Notch intracellular domain (NICD).

In some aspects, administering the vandefitemcel further comprises administering the vandefitemcel by intracerebral implantation.

In some aspects, the vandefitemcel can tonically release glutamate to the neurons of the brain of the subject after the vandefitemcel is implanted via intracerebral implantation.

In some aspects, administering the vandefitemcel further comprises injecting the vandefitemcel at multiple sites within the region of the brain.

In some aspects, administering the vandefitemcel further comprises administering the vandefitemcel stereotactically via a burr hole in the skull of the subject.

In some aspects, the region of the brain is the forebrain of the subject including at least one of the cerebrum, the thalamus, and the hypothalamus of the subject.

In some aspects, the region of the brain can be a site of injury or disease.

In some aspects, the region of the brain can be a hippocampus of the subject.

In some aspects, administering the vandefitemcel can further comprise administering the vandefitemcel by parenteral administration.

In some aspects, the vandefitemcel can be suspended in a sterile isotonic crystalloid solution.

In some aspects, administering the vandefitemcel can further comprise administering between about 1.0 million cells and 10.0 million cells of the vandefitemcel.

In some aspects, the vandefitemcel can be made by a method comprising providing a culture of MSCs; contacting the culture of MSCs with the polynucleotide encoding the NICD, wherein the polynucleotide does not encode a full-length Notch protein; selecting cells that comprise the polynucleotide; and further culturing the selected cells in the absence of selection for the polynucleotide.

In some aspects, the MSCs can be human bone marrow-derived cells.

In some aspects, the method can further comprise capturing functional magnetic resonance imaging (fMRI) scans of regions of the brain of the subject, selecting one region of the brain showing neuronal activity based on the fMRI scans, and administering the vandefitemcel to the one region of the brain.

In some aspects, a composition for increasing synapse formation in neurons is disclosed. The composition can comprise vandefitemcel in an amount between about 1.0 million cells and 10.0 million cells and one or more pharmaceutically acceptable excipients.

The vandefitemcel can be produced by modifying mesenchymal stem cells derived from human bone marrow.

In some aspects, the vandefitemcel can be made by a process comprising providing a culture of mesenchymal stem cells (MSCs); contacting the culture of MSCs with a polynucleotide encoding a Notch intracellular domain (NICD), wherein the polynucleotide does not encode a full-length Notch protein; selecting cells that comprise the polynucleotide; and further culturing the selected cells in the absence of selection for the polynucleotide.

In some aspects, the MSCs can be human bone marrow-derived cells.

In some aspects, the MSCs can be transiently-transfected with a plasmid vector comprising the polynucleotide encoding the NICD.

In some aspects, the one or more pharmaceutically acceptable excipients can comprise at least one of buffers, proteins, stabilizers, and preservatives.

In some aspects, the vandefitemcel can be suspended in a sterile isotonic crystalloid solution.

In some aspects, the one or more pharmaceutically acceptable excipients can comprise carriers or diluents.

In some aspects, a method of inducing, triggering, or causing the release of glutamate in a subject is disclosed. The method can comprise capturing functional magnetic resonance imaging (fMRI) scans of regions of the brain of the subject, selecting a region of the brain showing neuronal activity based on the fMRI scans, and administering vandefitemcel to the region of the brain selected. The vandefitemcel can be cells descended from mesenchymal stem cells (MSCs) transiently-transfected by a polynucleotide encoding a Notch intracellular domain (NICD). The vandefitemcel can release glutamate after being administered via intracerebral implantation.

In some aspects, a method of increasing synapse formation in neurons is disclosed. The method can comprise capturing functional magnetic resonance imaging (fMRI) scans of regions of the brain of the subject, selecting a region of the brain showing neuronal activity based on the fMRI scans, and administering vandefitemcel to the region of the brain selected comprising the neurons. The vandefitemcel can be cells descended from mesenchymal stem cells (MSCs) transiently-transfected by a polynucleotide encoding a Notch intracellular domain (NICD). The vandefitemcel can release glutamate to the neurons after being administered via intracerebral implantation.

### BRIEF DESCRIPTION OF THE DRAWINGS

FIGS. 1A-1D are graphs illustrating the electrophysiological activity and viability of separate cultures of vandefitemcel and iFbNs over an eight-week period using a multielectrode array (MEA) system.

FIG. 1E are raster plots illustrating electrophysiological activity and network burst development at four weeks and eight weeks for iFbN cell cultures at different cell densities.

FIG. 2A are reproductions of immunocytochemistry stains of a coculture of vandefitemcel and iPSC-derived forebrain neuron (iFbN) cells.

FIG. 2B is a graph illustrating the presence of vandefitemcel in cocultures of vandefitemcel and iFbN cells. The presence of vandefitemcel was confirmed by an increase in the resistance measured between the cells and the MEA electrodes.

FIG. 2C is a bar chart illustrating that at two weeks of coculture, an increase in neuronal excitability was detected in the iFbN and vandefitemcel coculture versus the iFbN culture alone based on the difference in the number of spikes observed.

FIG. 2D is a bar chart illustrating that at four weeks of coculture, the addition of vandefitemcel to the iFbN cells increased the number of network bursts in a cell density-dependent manner.

FIG. 2E are graphs illustrating an increase in glial fibrillary acidic protein (GFAP) cells in the coculture of iFbN cells with vandefitemcel compared to the iFbN control culture. FIG. 2E also illustrates that the 5-ethynyl-2′-deoxyuridine (EdU) populations were comparable for both the iFbN and vandefitemcel coculture and the iFbN control culture.

FIGS. 3A-3B are bar charts illustrating that after vandefitemcel was added to iFbN cells, the coculture of vandefitemcel and iFbN generated significantly more spikes and network bursts than the culture of iFbN cells alone. Also, FIGS. 3A-3B illustrate that cocultures of conditioned medium of vandefitemcel and iFbN also generated more spikes and network bursts than iFbN cells alone Furthermore, cocultures of human astrocytes and iFbN also generated more spikes and network bursts than iFbN cells alone.

FIG. 3C are raster plots illustrating electrophysiological activity and network burst development at four weeks of culture across MEA electrodes for iFbN cells alone and cocultures of vandefitemcel and iFbN, conditioned medium of vandefitemcel and iFbN, and human astrocytes and iFbN.

FIG. 4A are reproductions of various immunocytochemistry (ICC) stains of a culture of iFbNs at thirty days of culture maturation.

FIG. 4B is a bar chart illustrating that, at day three of coculture, vandefitemcel promoted high levels of spike activity in glutamatergic neurons, when compared to spike activity in either the glutamatergic neurons alone or the glutamatergic neurons and human astrocytes cocultures.

FIG. 4C is a graph illustrating that the spike activity in glutamatergic neurons promoted by vandefitemcel kept increasing for two weeks while human astrocytes (hA) slightly promoted spike activity in glutamatergic neurons starting at week three.

FIG. 4D is a graph illustrating that vandefitemcel induced significant network burst activity in the first week of coculture with glutamatergic neurons while glutamatergic neurons in coculture with human astrocytes (GluN+hA) did not show network bursts until week four.

FIG. 4E are raster plots illustrating the electrophysiological activity of cocultures of glutamatergic neurons and vandefitemcel over time and progressive network burst development across electrodes on the MEA system.

FIGS. 4F and 4G are graphs illustrating that vandefitemcel promoted spikes and network bursts of GABAergic neurons in week two and week four of coculture when compared to GABAergic neurons alone or in cocultures of GABAergic neurons and human astrocytes.

FIG. 4H are raster plots illustrating the electrophysiological activity and network burst development of cocultures of GABAergic neurons and vandefitemcel over time and progressive network burst development across electrodes on the MEA system.

FIG. 5A is a bar chart illustrating that at two days of culture, the increased levels of conditioned medium (CM) glutamate were cell density-dependent in cultures of both vandefitemcel and human astrocytes (hA), but the levels of glutamate in CM of vandefitemcel were about 10-fold higher than that in the CM of hA.

FIG. 5B is a graph illustrating that, at five days of culture, the levels of glutamate in CM of vandefitemcel increased 4.9-fold while the levels of glutamate in CM of hA increased 2.7-fold.

FIG. 5C is a bar chart illustrating that after incubation with 5 μM and 50 μM of a glutaminase inhibitor (CB-839), the levels of glutamate in the CM of vandefitemcel were reduced 1.5-fold and 2.0-fold, respectively, compared to the control of vandefitemcel incubated with dimethylsulfoxide (DMSO).

FIG. 5D is a bar chart illustrating that incubation with CB-839 did not alter the viability of vandefitemcel.

FIGS. 5E and 5F are bar charts illustrating that after one week of coculture, the inhibition of glutaminase in vandefitemcel significantly reduced spike activity and network bursts in the cocultures of glutamatergic neurons and vandefitemcel compared to the control culture of vandefitemcel incubated with DMSO.

FIGS. 5G and 5H are bar charts illustrating that astrocytes reduced the number of spikes and network bursts that would have otherwise been induced by vandefitemcel on glutamatergic neurons.

FIG. 5I is a schematic diagram showing vandefitemcel promoting an increase in neuronal activity and synaptic plasticity via tonic glutamate release.

### DETAILED DESCRIPTION

The below terms are defined as follows for purposes of this disclosure.

### Definitions

The terms “administration” and “administering” refer to the physical introduction of a composition comprising a therapeutic agent to a subject, using any of the various methods and delivery systems known to those skilled in the art. For example, routes of administration for vandefitemcel can include intracerebral, intrathecal, or other parenteral routes of administration, for example by injection or infusion. The phrase “parenteral administration” as used herein means modes of administration other than enteral and topical administration, usually by injection and infusion. Administering can also be performed, for example, once, a plurality of times, and/or over one or more extended periods, and can be a therapeutically effective dose or a subtherapeutic dose.

The terms “implantation” and “transplantation” are used to denote the introduction of exogenous cells (e.g., vandefitemcel or SB623 cells) into a subject or patient. Exogenous cells can be autologous (i.e., obtained from the subject) or allogeneic (i.e., obtained from an individual other than the subject).

An “isotonic crystalloid solution” is a solution containing water-soluble electrolytes. Such a solution contains the same amount of electrolytes as plasma.

“Mesenchymal cells” refer to cells of mesenchymal tissue (e.g., chondroblasts, chondrocytes, osteoblasts, osteocytes, adipocytes) and their precursors and include, for example, fibroblasts (e.g., human foreskin fibroblasts), MSCs (as defined herein) and cells derived from MSCs such as, for example, vandefitemcel, as defined herein.

“MSCs” (“mesenchymal stem cells”) refer to adherent, non-hematopoietic pluripotent cells obtained from bone marrow. These cells are variously known as mesenchymal stem cells, mesenchymal stromal cells, marrow adherent stromal cells, marrow adherent stem cells and bone marrow stromal cells. MSCs can also be obtained from, e.g., umbilical cord blood, adipose tissue, dental pulp, Wharton's jelly, and various types of connective tissue. MSCs can be obtained by selecting (e.g., by growth in culture) adherent cells (i.e., cells that adhere to tissue culture plastic) from bone marrow. To obtain MSC populations having a sufficient number of cells for use in therapy, populations of adherent cells are expanded in culture after selecting for adherence. Expansion in culture also enriches MSCs, since contaminating cells (such as monocytes) do not proliferate under the culture conditions. Exemplary disclosures of MSCs are provided in U.S. Patent Publication No. 2003/0003090 and the literature [8]. Methods for isolating and purifying MSCs can be found, for example, in U.S. Pat. No. 5,486,359 and the literature [9, 10]. Human MSCs are commercially available (e.g., BioWhittaker, Walkersville, Md.) or can be obtained from donors by, e.g., bone marrow aspiration, followed by culture and selection for adherent bone marrow cells. Sec, e.g., WO 2005/100552. MSCs can also be isolated from umbilical cord blood [11, 12]. Additional sources of MSCs include, for example, adipose tissue, dental pulp, and Wharton's jelly.

“Network burst” refers to a collective or synchronous/near-synchronous behavior of neurons comprised of alternating periods of relatively fast spiking or spike behavior followed by inactivity. Also referred to as population bursting.

“Neuronal spike” or “spike” refers to an action potential or a brief electrical impulse that can be captured or recorded when neurons fire. Spikes can be picked up by electrodes such as electrodes of a microelectrode array.

A “neurodegenerative disorder,” “neurological disorder,” or “neurological disease” refers to disorders or diseases in which cells of the central nervous system (CNS) stop working, die, or are compromised in some manner. These types of diseases or disorders can include, but are not limited to, Alzheimer's disease, Parkinson's disease, Huntington's disease, amyotrophic lateral sclerosis (ALS), traumatic brain injury (TBI), ischemic stroke, hemorrhagic stroke, chronic stroke, or spinal cord injury.

The “Notch protein” (e.g., Notch 1 protein) is a transmembrane receptor, found in all metazoans, that influences cell differentiation through intracellular signaling. Contact of the Notch extracellular domain (e.g., the extracellular domain of the Notch 1 protein) with a Notch ligand (e.g., Delta, Serrate, Jagged) results in two proteolytic cleavages of the Notch protein, the second of which is catalyzed by a y-secretase and releases the Notch intracellular domain (NICD) into the cytoplasm. In the mouse Notch protein, this cleavage occurs between amino acids gly1743 and val1744. The NICD translocates to the nucleus, where it acts as a transcription factor, recruiting additional transcriptional regulatory proteins (e.g., MAM, histone acetylases) to relieve transcriptional repression of various target genes (e.g., Hes 1). Additional details and information regarding Notch signaling can be found in the literature [13, 14, 15].

The term “pharmaceutically acceptable excipient” refers to an excipient for administration of a pharmaceutical agent. These “excipients” refer to any substance other than the active pharmaceutical agent. Examples of pharmaceutically acceptable excipients can include, but are not limited to, buffers, proteins, carbohydrates, oleochemicals, petrochemicals, stabilizers, preservatives, fillers, diluents, binders, viscosity agents, coatings, disintegrants, colorants, and lubricants.

The terms “vandefitemcel,” vandefitemcel cells,” “SB623,” and “SB623 cells” refer to populations of cells obtained following transient expression of an exogenous Notch intracellular domain (NICD) in MSCs. For example, a population of vandefitemcel or SB623 cells can be obtained by transient transfection of MSCs with a plasmid vector comprising sequences encoding a NICD (e.g., from the human Notch 1 protein) but not encoding a full-length Notch protein followed by selection (e.g., with G418). The selected cells can be further cultured in a standard culture medium, optionally supplemented with a serum, in the absence of any added growth factors or differentiation factors (other than those which may be present in the serum, if serum is present in the culture medium). Vandefitemcel can be derived from human (allogeneic) bone marrow MSCs by transient transfection of human bone marrow MSCs with NICD (e.g., the human Notch 1 intracellular domain (NICD1)), followed by selection, and subsequent expansion. This process produces a cell population that is different than the parental MSCs [5, 6, 7]. Vandefitemcel or SB623 cells have also been referred to as descendants of NICD transiently-transfected MSCs (“DNTT-MSCs”).

The term “transiently-transfected” refers to methods of introducing foreign DNA or RNA into cells for temporary expression of the introduced gene(s). Transient transfection influences short-term gene expression, lasting for a few hours to several days.

The terms “tonic release of glutamate” and “tonically release glutamate” refer to the non-vesicular release of glutamate from neurons or glial cells into the extracellular space. Tonic glutamate release can help maintain or modulate a subject's baseline neuronal activity and help maintain or modulate a subject's synaptic plasticity.

“Therapeutically effective” means the amount of an agent required to provide a meaningful patient benefit or promote disease regression evidenced by a decrease in severity of disease symptoms, an increase in frequency and duration of disease symptom-free periods, or a prevention of impairment or disability due to the disease affliction. The therapeutically effective amount of an agent can be evaluated using a variety of methods known to the skilled practitioner such as a practitioner in the field of neurology.

### Preparation of Vandefitemcel

The vandefitemcel administered are allogeneic cells descended from mesenchymal stem cells transiently-transfected by a polynucleotide encoding a Notch intracellular domain (NICD). The cells can be made by a method comprising providing a culture of the mesenchymal stem cells, contacting the culture of mesenchymal stem cells with the polynucleotide encoding an NICD (where the polynucleotide does not encode a full-length Notch protein), selecting cells that comprise the polynucleotide, and further culturing the selected cells in the absence of selection for the polynucleotide. The mesenchymal stem cells can be human bone marrow-derived cells.

As previously discussed, vandefitemcel can be obtained from marrow-adherent stromal cells, also known as MSCs, by transiently expressing the intracellular domain of the Notch protein in the MSCs. Transient expression of the Notch intracellular domain (e.g., the NICD from the human Notch 1 protein) in an MSC can be sufficient to convert a population of MSCs into a population of vandefitemcel. Additional treatment with growth and/or differentiation factors is not required. Thus, a population of MSCs can be converted to a population of vandefitemcel by transient transfection of MSCs with a plasmid vector comprising sequences encoding an NICD (but not encoding full-length Notch protein), followed by selection for cells comprising the vector and further culture of the selected cells in serum-containing medium, in the absence of exposure to additional growth and/or differentiation factors. See, for example, U.S. Pat. Nos. 7,682,825; 8,945,919; and WO 2009/023251; the contents of which are incorporated herein by reference in their entireties for the purposes of describing isolation of mesenchymal stem cells and conversion of mesenchymal stem cells to vandefitemcel (also referred to as “neural precursor cells” and “neural regenerating cells” in those documents).

In this disclosure, any polynucleotide encoding a Notch intracellular domain (e.g., a plasmid vector) can be used, and any method for the selection and enrichment of transfected cells can be used. For example, MSCs can be transfected with a vector containing sequences encoding a Notch intracellular domain (e.g., the human Notch 1 intracellular domain) and also containing sequences encoding a selection marker (e.g., drug resistance; e.g., resistance to G418). In some instances, two plasmid vectors, one containing sequences encoding a Notch intracellular domain and the other containing sequences encoding a drug resistance marker, can be used for transfection of MSCs. In these instances, selection is achieved, after transfection of a cell culture with the vector or vectors, by adding a selective agent (e.g., G418) to the cell culture in an amount sufficient to kill cells that do not comprise the vector but spare cells that do. Absence of selection entails the removal of said selective agent or reduction of its concentration to a level that does not kill cells that do not comprise the vector. Following selection (e.g., for seven days) the selective agent can be removed and the cells can be further cultured (e.g., for two passages) in serum-containing culture medium.

It is also possible, depending on the nature of the selection marker and/or the concentration of the selective agent used, that not every cell that lacks a vector encoding a selection marker will be killed during the selection process. For example, a selective agent may inhibit growth of a cell not comprising the selection marker and, after removal of the selective agent, the cell may recover and resume growth.

Preparation of vandefitemcel thus involves transient expression of an exogenous Notch intracellular domain in an MSC. To this end, MSCs can be transfected with a plasmid vector comprising sequences encoding a Notch intracellular domain (e.g., the human Notch 1 intracellular domain) wherein said sequences do not encode a full-length Notch protein. All such sequences are known and readily available to those of skill in the art [14].

Similar information is available for Notch proteins and nucleic acids from additional species, including rat, Xenopus, Drosophila, and human [16, 17]. Additional information can also be found in NCBI Reference Sequence No. NM_017167, SwissProt P46531, SwissProt Q01705, and GenBank CAB40733. The foregoing references are incorporated by reference in their entireties for the purpose of disclosing the amino acid sequence of the full-length Notch protein and the amino acid sequence of the Notch intracellular domain in a number of different species.

In some instances, vandefitemcel can be prepared by introducing, into MSCs, a nucleic acid comprising sequences encoding a Notch intracellular domain such that the MSCs do not express exogenous Notch extracellular domain. Such can be accomplished, for example, by transfecting MSCs with a plasmid vector comprising sequences encoding a Notch intracellular domain wherein said sequences do not encode a full-length Notch protein.

Additional details on the preparation of vandefitemcel or SB623 cells, and methods for making cells with properties similar to those of vandefitemcel or SB623 cells which can be used in the methods disclosed herein, can be found in the literature [18] and U.S. Pat. Nos. 7,682,825; 8,945,919; and 9,441,199, the contents of which are incorporated herein by reference in their entireties for the purposes of describing alternative methods for the preparation of, vandefitemcel, and for providing methods for making cells with properties similar to those of vandefitemcel.

The vandefitemcel used for the experiments disclosed herein were stored in liquid nitrogen. For cocultures, except where indicated, the vandefitemcel cells were thawed, washed, and plated directly. For glutaminase inhibition assays, vandefitemcel (or SB623) cells were cultured after thawing in minimum essential medium-alpha (α-MEM) (from CORNING, Corning, NY) supplemented with 10% of fetal bovine serum (FBS). In cocultures with neurons or for glutamate assay, vandefitemcel (or SB623) cells were kept in BrainPhys™ medium supplemented with NeuroCult™ SM1 neuronal supplement. To assess the levels of glutamate release by the vandefitemcel cells into conditioned medium, Gibco GlutaMAX™ (100×; 200 mM L-alanyl-L-glutamine from Thermo Fisher Scientific, Waltham, MA) was added (final concentration 2 mM) to vandefitemcel cells cultured in BrainPhys™ medium supplemented with NeuroCult™ SM1 neuronal supplement and levels of glutamate were assessed after two days.

### Transfection

Methods for introduction of exogenous DNA into cells (i.e., transfection), and selection of transfected cells, are known in the art [19, 20].

### Preparation of iPSC-Derived Forebrain Neurons (iFbNs)

Human episomal iPSCs (e.g., Gibco™ A18945 hiPSC from Thermo Fisher Scientific, Waltham, MA) were grown using a StemFlex™ Medium Kit (from Thermo Fisher Scientific) in six-well tissue culture plates pre-coated with Geltrex™ hESC-Qualified (from Thermo Fisher Scientific) and passaged with ReLeSR™ (from Stem Cells Technologies, Vancouver, Canada). To generate forebrain neurons, iPSCs were differentiated into neural progenitor cells using STEMdiff™ Forebrain Neuron Differentiation Kit (from Stem Cells Technologies, Vancouver, Canada). After a single cell dissociation procedure, iPSCs were seeded on six-well tissue culture plates pre-coated with 15 μg/mL of poly-L-ornithine (from Sigma-Aldrich, St. Louis, MO) and 20 μg/mL of laminin (Sigma-Aldrich, St. Louis, MO) and differentiated using a monolayer culture system in StemDiff™ Neural Induction Medium plus SMADi (both from Stem Cells Technologies, Vancouver, Canada) following neural induction procedures (Stem Cells Technologies, Vancouver, Canada). Neural progenitor cells were passaged to a new six-well tissue culture plate pre-coated with 15 μg/mL of poly-L-ornithine and 20 μg/mL of laminin and then differentiated into forebrain neurons using the STEMdiff™ Forebrain Neuron Differentiation Kit for one week. After differentiation, forebrain neurons were matured using the STEMdiff™ forebrain neuron maturation kit (from Stem Cells Technologies, Vancouver, Canada) for two weeks. Forebrain neuronal cultures were then kept in BrainPhys™ medium supplemented with NeuroCult™ SM1 neuronal supplement (from Stem Cells Technologies, Vancouver, Canada). All cells were grown in humidified sterile environments at 37°° C. and 5% CO₂.

### Preparation of Human Glutamatergic Neurons, Human GABAergic Neurons, and Human Astrocytes

Human glutamatergic neurons (e.g., iCell GlutaNeurons™) and human GABAergic neurons (e.g., iCell GABAneurons™) were obtained from FUJIFILM Cellular Dynamics FUJIFILM Cellular Dynamics Inc. (Madison, WI) and directly cultured on multielectrode array (MEA) plates/wells pre-coated with 15 μg/mL of poly-L-ornithine (from Sigma-Aldrich, St. Louis, MO) and 20 μg/mL of laminin (from Sigma-Aldrich, St. Louis, MO) in BrainPhys™ medium supplemented with NeuroCult™ SM1 neuronal supplement. Human fetal-derived astrocytes (from ScienceCell Research Laboratories Carlsbad, CA) were seeded on T-75 flasks pre-coated with 10 μg/mL of poly-L-lysine (from Sigma-Aldrich, St. Louis, MO) and expanded in astrocytes medium supplemented with 2% fetal bovine serum (FBS) and 1% of astrocytes supplement (all from Science Cell Research Laboratories). Human astrocytes were kept in BrainPhys™ medium supplemented with NeuroCult™ SM1 neuronal supplement prior to cocultures with neurons or for use in glutamate assays.

### Multielectrode Array (MEA) System

Wells/plates of Cyto View MEA 24 (from Axion Biosystems, Atlanta, GA) were treated with 30-50 μL of 15 μg/mL of poly-L-ornithine for two hours or overnight at 37° C. and 5% CO₂. The wells were washed twice with phosphate-buffered saline (PBS) and once with Dulbecco's Modified Eagle Medium/Nutrient Mixture F-12 and 4-(2-Hydroxyethyl) piperazine-1-cthane-sulfonic acid (DMEM/F12-HEPES) (from Stem Cells Technologies, Vancouver, Canada). Wells were coated with 30-50 μL of 20 μg/mL of laminin (from Sigma-Aldrich, St. Louis, MO) for two hours or overnight at 37° C. and 5% CO₂. Laminin was removed and neurons were immediately seeded and incubated for 30-45 minutes at 37° C. and 5% CO₂. Then fresh medium was added to reach a volume of 400 μL per well. Half of the medium volume was changed every two to three days. Forebrain neurons (iFbNs), 3×10⁴ or 5×10⁴ cells per well, were cultured in BrainPhys™ supplemented with maturation supplements (from Stem Cells Technologies, Vancouver, Canada) for two weeks. After two weeks, the medium was changed to BrainPhys™ medium supplemented with NeuroCult™ SM1 neuronal supplement. To coculture vandefitemcel cells or human astrocytes with iPSC-derived forebrain neurons (iFbNs), the medium was transferred into a 24-well plate leaving about 10-20 μL in the inner well of the MEA plates. The vandefitemcel (or SB623) cells or astrocytes were added in cell solution of 10-20 μL and incubated for 45 minutes at 37° C. and 5% CO₂. After 30-45 minutes of incubation, 1:1 fresh medium and saved conditioned medium were added to reach a volume of 400 μL per well. Half of the medium volume was changed every three or four days.

Glutamatergic or GABAergic neurons, 9×10⁴ cells per well or 1×10⁵ cells per well, were plated in 25 μL volume cell in BrainPhys™ medium supplemented with NeuroCult™ SM1 neuronal supplement and incubated for 30-45 minutes at 37° C. and 5% CO₂. For coculturing, vandefitemcel (or SB623) cells or astrocytes were cocultured by adding 10-20 μL of cell solution with 1.5×10⁴ cells per well following a new incubation for 45 minutes at 37° C. and 5% CO₂. Fresh medium was added to reach a volume of 400 μL per well and cells incubated at 37° C. and 5% CO₂. Half of the medium volume was changed every three or four days. To isolate the presynaptic activity on MEAs with iFbNs after 50 days of culture, 50 μM of the broad glutamate receptor antagonist Kynurenic acid (from R&D Systems, Minneapolis, MN) was added at 90 seconds from the start of the recording. Spike activity was assessed to detect the blockage of glutamate receptor activity.

MEA recordings were performed at 37° C. and 5% CO₂ using Maestro Edge (from Axion Biosystems, Atlanta, GA). The medium was changed at least two hours before the recordings. Before recordings, levels of CO₂ were adjusted to 5%. The duration of each recording was five minutes using AxIS Navigator 3.6.2 with configuration module Neural Real-Time; Spontaneous+Viability from Axion Biosystems with minimum spike rate for active electrodes of five spikes/minute.

### Flow Cytometry

iPSC-derived forebrain neuronal cultures were dissociated from multielectrode wells into single cell suspension(s). For this, cultures were washed once with phosphate-buffer saline (PBS) (from Thermo Fisher Scientific, Waltham, MA) and incubated with TrypLE™ express (from Thermo Fisher Scientific, Waltham, MA) for 5 minutes at 37° C. and 5% CO₂. Cells were recovered in BrainPhys™ medium and washed 2× followed by 30 minutes of fixation with 500 μL of eBioscience™ IC fixation buffer (from Thermo Fisher Scientific, Waltham, MA) in PBS at room temperature. After fixation, the cells were washed twice for 5 minutes at 300 g with 2 mL of eBioscience™ Permeabilization Buffer (from Thermo Fisher Scientific, Waltham, MA). The cells were incubated with a blocking solution containing 5% normal goat serum (from Jackson ImmunoResearch Inc., West Grove, PA)+2% bovine serum albumin (BSA) in eBioscience™ Permeabilization buffer for 15 minutes at room temperature using an orbital shaker protected from light. Without washing the cells, the conjugated antibody was added directly to cells for detection of intracellular antigen and incubated for minutes at room temperature using an orbital shaker and protected from light. The conjugated antibodies used were REA Control Antibody REAfinity™ and GFAP Antibody REAfinity™ (both from Miltenyi Biotec, San Jose, CA). The cells were washed two times for 5 minutes at 300 g centrifugation with eBioscience™ Permeabilization buffer and resuspended in eBioscience™ Flow Cytometer Staining Buffer (from Thermo Fisher Scientific, Waltham, MA). For the proliferation assay, Click-iT® 5-cthynyl-2′-deoxyuridine (EdU) Flow Cytometry Assay Kits (from Thermo Fisher Scientific, Waltham, MA) were used. EdU was added for 24 hours. Flow cytometry was performed using MACSQuant® Analyzer 10 Flow Cytometer and data were analyzed using MACSQuantify™ (both from Miltenyi Biotec, San Jose, CA).

### Immunocytochemistry Stains

iFbNs and vandefitemcel (or SB623) cells were cocultured in Nunc™ Lab-Tek™ II CC2 chamber slides previously coated with poly-L-ornithine and laminin. Cells were washed with PBS (from Thermo Fisher Scientific, Waltham, MA), followed by 15 minutes of incubation with 4% paraformaldehyde at room temperature. For staining, the samples were blocked with 5% normal goat serum (from Jackson ImmunoResearch Laboratories Inc., West Grove, PA) in PBS with a nonionic surfactant (e.g., 0.3% Triton X-100 from Sigma-Aldrich) for 30 min at room temperature. The primary antibodies used were rabbit anti-GFP (1:200;from Abcam, Fremont, CA), rabbit anti-GFAP (1:1000; from Agilent DAKO), rabbit anti-Olig2 (1:100; from Cell Signaling, Danvers, MA), chicken anti-MAP2 (1:1000; from Abcam, Fremont, CA), rabbit anti-synaptophysin (1:500; from Abcam, Fremont, CA), mouse anti-VGLUT1 (1:1000) and rabbit anti-GABA (1:100; from Sigma-Aldrich, St. Louis, MO). Cells were washed three times with PBS with nonionic surfactant (e.g., 0.3% Triton X-100 from Sigma-Aldrich, St. Louis, MO) and incubated with respective secondary antibodies overnight at 4° C. The secondary antibodies used were Alexa Fluor 488-conjugated goat anti-rabbit (1:1000; Invitrogen, Waltham, MA), Alexa Fluor™ 546-conjugated goat anti-mouse (1:1000; from Invitrogen, Waltham, MA), Alexa Fluor™ 647-conjugated goat anti-chicken (1:1000; from Invitrogen, Waltham, MA). Followed by washing three times with PBS with a nonionic surfactant (e.g., 0.3% Triton X-100 for 5 minutes each. The slides were mounted with Fluoromount-G™ mounting medium, with 4′,6-diamidino-2-phenylindole (DAPI) (from Thermo Fisher Scientific, Waltham, MA). Fluorescence images were recorded using a digital camera attached to a fluorescence microscope (from Leica DMi8, Wetzlar, Germany). Image processing was performed by Fiji-ImageJ software (from National Institute of Health; NIH). Antibody specificity and sample backgrounds were tested by using only secondary antibodies for each slice stained.

### Determination of Glutamate Concentration

The concentration of glutamate in conditioned medium was determined using the Glutamate Assay Kit from Sigma-Aldrich (St. Louis, MO) and following the protocol provided by the manufacturer. Cells were seeded at different cell densities on a 24-well or a 96-well plate pre-coated with poly-D-lysine and laminin in BrainPhys™ medium supplemented with NeuroCult™ SM1 neuronal supplement. Cells were kept at 37° C. and 5% CO₂. Approximately 50 μL of conditioned medium was collected at different time points. Media collected were stored at −20° C. until measurement of glutamate concentrations. 50 μL of conditioned medium for each sample was used to measure the concentration of glutamate. The concentration of glutamate in conditioned medium was measured during five days of culture in time course experiments. Absorbance was measured after 30 minutes of incubation on SpectraMax® iD3 Multi-mode Microplate Reader (from Molecular Devices, San Jose, CA).

### Viability Assays

Approximately 50 μL of conditioned medium (CM) was used to determine the glutamate concentration and cell viability. For this assay, PrestoBlue™ high-sensitivity (HS) Cell Viability Reagent (from Thermo Fisher Scientific, Waltham, MA) was used and the reaction absorbance of each sample was read after 2 hours of incubation following the manual procedure provided by the manufacturer.

### Inhibition of Glutaminase

Vandefitemcel (or SB623) cells were cultured with α-MEM supplemented with 10% fetal bovine serum (FBS) for 16-24 hours at 37° C. and 5% CO₂. Next, vandefitemcel (or SB623) cells were treated with two different concentrations of the glutaminase inhibitor Telaglenastat (CB-839; from Selleckchem, Houston TX): 5 μM and 50 μM for two or three days. vandefitemcel (or SB623) cells were washed two times and cryopreserved for a subsequent coculture with glutamatergic neurons for the MEA experiments. In parallel, 1.0×10⁴ vandefitemcel (or SB623) cells were incubated with CB-839 for 2 days at 37° C. and 5% CO₂. Cells were washed two times with PBS and cultured in BrainPhys™ medium supplemented with NeuroCult™ SM1 neuronal supplement for three days at 37° C. and 5% CO₂. The levels of glutamate in conditioned medium were assessed.

### Statistical Analysis

Statistical analyses were performed using GraphPad® Prism version 9.4.1 (from GraphPad Software, San Diego, California, USA). Correlation analysis was evaluated using Spearman's correlation coefficient with a 95% confidence interval. The significance of differences between groups was determined by one-way ANOVA followed by Holm-Sidak's post hoc test, paired t-test, or Mann-Whitney test (two-tailed), as indicated in the figure legends. Comparisons of multiple groups and time points were performed using analysis of variance (ANOVA) followed by Tukey's or Sidak's post hoc tests, or mixed-effects models with Geisser-Greenhouse correction and Tukey's post hoc tests. All tests were two-tailed unless otherwise specified.

### Electrophysiological Activity and Viability of Vandefitemcel and iFbNs

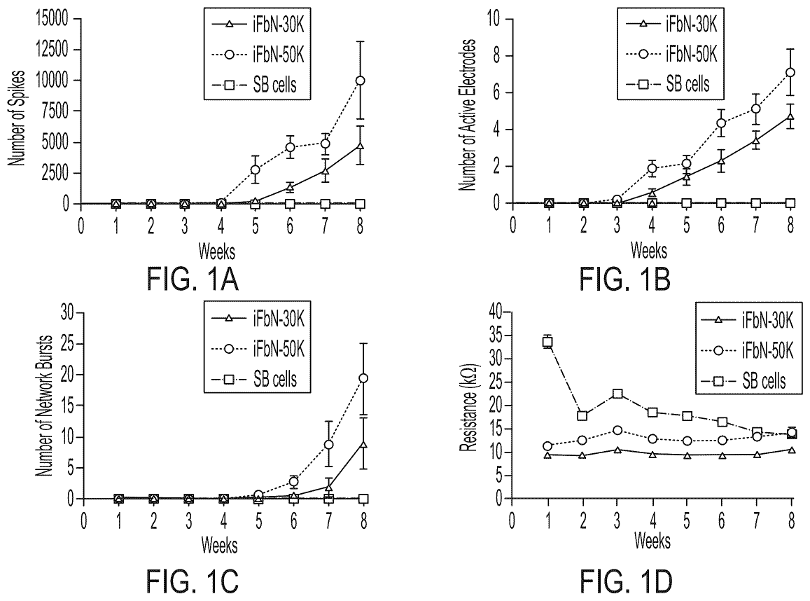

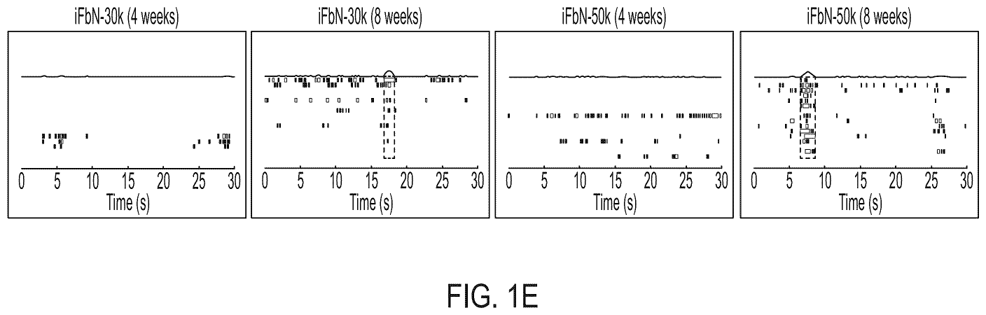

To determine whether vandefitemcel (or SB623 cells) exhibit neuron-like electrophysiological behavior, the electrophysiological activities of vandefitemcel (or SB623 cells) and iFbNs were evaluated as single/separate cultures in serum-free neurophysiological conditions using the Axion Biosystem MEA system [21]. As shown in FIGS. 1A-1C, during eight weeks of culture, vandefitemcel (or SB623 cells) at a cell density of 2.5×10⁴ cells did not exhibit any spontaneous neuron-like electrophysiological activity as evidenced by the number of spikes, active electrodes, and network bursts. However, around three weeks of culture, iFbNs (at cell densities of both 3.0×10⁴ cells and 5.0×10⁴ cells) showed spontaneous neuron-like electrophysiological activity as evidenced by the number of spikes and active electrodes (sec FIGS. 1A and 1B). iFbNs also exhibited spontaneous neuronal oscillations in the form of network bursts starting at around four to five weeks of cell culture (see FIG. 1C). When iFbN cells were plated at a higher cell density, they exhibited earlier and more pronounced neuronal oscillations when compared to cells plated at a lower cell density, although the difference was not statistically significant (see FIG. 1E).

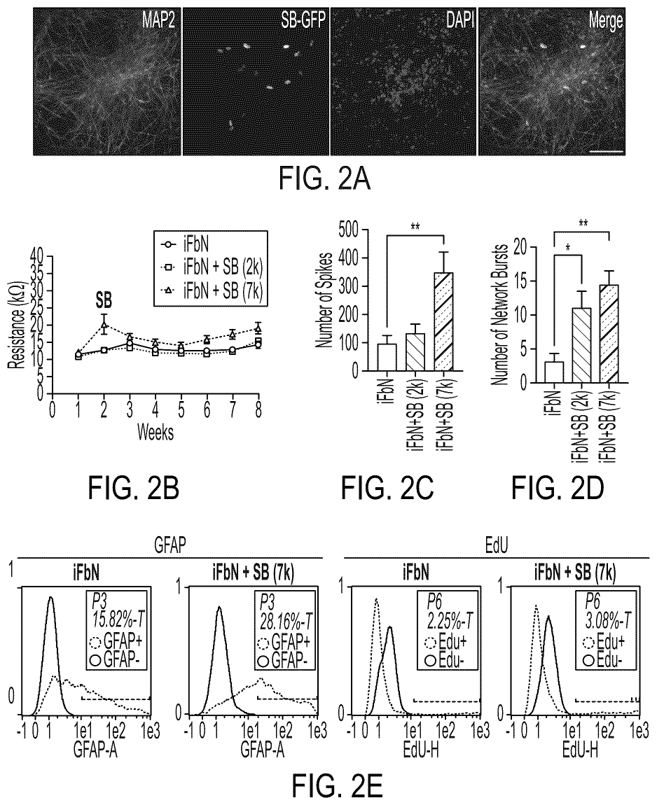

The viability of vandefitemcel (or SB623 cells) and iFbN cells were evaluated in neurophysiological conditions using the impedance module available within the Axion Biosystem MEA system. Resistance (in ohms, (2) was measured between the cells and the electrodes. In suspension, vandefitemcel (or SB623 cells) were large (around 17.2 μm diameter) and 100% confluent, covering all 16 electrodes. In contrast, iFbN cells in suspension were smaller (around 10.2 μm diameter) and formed cell clusters interconnected with cellular projections when attached to MEA wells. These particular hallmarks distinguish vandefitemcel (or SB623 cells) and iFbN cells and resulted in significantly different resistances, as seen in the first week of culture (see FIG. 1D). Although the resistance assessed using the MEA system indicated that vandefitemcel (or SB623 cells) survived in BrainPhys™ medium, there was a significant reduction in resistance in the first three weeks of culture (32.6%), and a continuous drop that reached 58.8% by the eighth week of culture. On the other hand, it was observed that in both low and high cell densities, iFbN cells exhibited increased resistance in the second and third weeks (14% and 28.9% respectively), compared to the resistance observed in the first week in culture (see FIG. 1D). In the third week, and upon withdrawn of maturation supplements, the resistance in iFbN cultures slightly dropped and then remained stable until five-weeks of culture, when the resistance started to increase again. This increased resistance seen in the iFbN cell cultures suggests the presence of progenitor cells in these neuronal cultures. Indeed, at five weeks of culture, 2.25% of iFbN cell cultures were EdU-positive after 24 hours of EdU labeling (see FIG. 2C). Despite the increase in resistance in iFbN cell cultures, there was no correlation between resistance and spike counts at low or high iFbN cell seeding (Spearman's correlation, r=0.0665 and r=−0.1189, respectively) indicating that the increase in neuronal activity could not be explained by more cells occurring within the MEA system.

FIG. 1E are raster plots of iFbN cells plated at cell densities of 3.0×10⁴ cells and 5.0×10⁴ cells representing the electrophysiological activity of such cells in the form of spikes and network bursts. Single spikes are represented in the plots by black boxes, electrode bursts are represented in the plots by white boxes, and network bursts are represented by broken-line boxes.

Taken together, vandefitemcel (or SB623 cells) survived in the MEA system but did not exhibit neuron-like electrophysiological activities, while iFbN cells exhibited some electrophysiological activities as evidenced by increasing spike counts, active electrodes, and neuronal network bursts in the Axion Biosystem MEA system.

### EXAMPLES

The examples below are given so as to illustrate the practice of various embodiments of the present disclosure. They are not intended to limit or define the entire scope of this disclosure. It should be appreciated that the disclosure is not limited to the particular embodiments described and illustrated herein but includes all modifications and variations falling within the scope of the disclosure as defined in the appended embodiments.

### Example 1: Coculturing Vandefitemcel With iFbN Cells Results in Increased Neuronal Activity and Synapse Formation in a Cell Density-Dependent Manner

FIGS. 2A-2E illustrate that vandefitemcel (or SB623 cells, shown as SB in FIGS. 2A-2E) promotes neuronal activity/excitability and synapse formation while in coculture with iFbN cells. FIG. 2A are immunocytochemistry (ICC) stains of a coculture of vandefitemcel and iFbN cells where the iFbN cells express the neuronal marker MAP2, the vandefitemcel (or SB623cells) express green fluorescent protein (GFP), and cell nuclei are stained with 4′,6-diamidino-2-phenylindole (DAPI). iFbN cells were plated at a cell density of 5×10⁴ cells and after 14 days of maturation, vandefitemcel were added at two cell densities (2.0×10³ cells and 7.0×10³ cells). As shown in FIG. 2A, after thirty days of coculture, vandefitemcel cells (shown as SB-GFP) were detected within a culture of MAP2 positive forebrain neurons. FIG. 2B illustrates that the presence of the vandefitemcel (or SB623 cells, indicated as “SB” in the figures) was confirmed by an increase in the resistance measured between the cells and the MEA electrodes measured only one day after the beginning of the coculture at the higher cell density (7.0×10³ cells) of vandefitemcel. At the higher cell density (7.0×10³ cells) of vandefitemcel, an increase in resistance was observed starting in the fourth week of coculture.

To evaluate whether vandefitemcel affected the neuronal electrical activity of iFbNs, the number of spikes in the iFbN control culture (shown as “iFbN” in FIG. 2C) was compared to the number of spikes in the cocultures of iFbN and vandefitemcel of different cell densities (shown as “iFbN+SB (2k)” for the coculture of iFbN cells with 2.0×10³ cells of 24 vandefitemcel and shown as “iFbN+SB (7k)” for the coculture of iFbN cells with 7.0×10³ cells of vandefitemcel in FIG. 2C). FIG. 2C illustrates that at two weeks of coculture, an increase in neuronal excitability was detected in the cocultures of iFbN and vandefitemcel coculture versus the iFbN culture based on the difference in the number of spikes observed. As shown in FIG. 2C, the number of spikes, or the degree of neuronal excitability, increased in a cell density-dependent manner. The difference in the number of spikes between the iFbN control culture and the iFbN+SB (7k) coculture was statistically significant (one-way ANOVA Holm-Sidak, **p<0.005).

To determine whether the increased neuronal activity promoted by the vandefitemcel (or SB623 cells) also increased synapse formation, the number of network bursts was also counted for the culture of iFbN cells (shown as “iFbN” in FIG. 2D), the coculture of iFbN cells with 2.0×10³ cells of vandefitemcel (shown as “iFbN+SB (2k)”), and the coculture of iFbN cells with 7.0×10³ cells of vandefitemcel (shown as “iFbN+SB (7k)”). FIG. 2D illustrates that at four weeks of coculture, the addition of vandefitemcel to the iFbNs also increased the number of network bursts in a cell density-dependent manner. The spike and network burst counts continued to rise over the next eight weeks of coculture, remaining distinguishable from the control of just iFbN cells. The difference in the number of network bursts between the iFbN control culture and either of the iFbN+SB cocultures was statistically significant (one-way ANOVA Holm-Sidak, *p<0.05; **p<0.005).

It was previously reported that vandefitemcel (or SB623 cells) promoted neuropoiesis in an embryonic rat neural culture [22, 23]. FIG. 2E shows an increase in glial fibrillary acidic protein (GFAP+) cells in the coculture of iFbN cells with 7.0×10³ cells of vandefitemcel (shown as “iFbN+SB (7k)” in FIG. 2E) compared to the iFbN control culture (shown as “iFbN” in FIG. 2E). FIG. 2E is a flow cytometer analysis showing that the GFPA+ population in the iFbN+SB (7k) coculture was ˜28.2% compared to ˜15.82% in the iFbN control culture. The EdU+ populations were comparable for both the iFbN+SB (7k) coculture and the iFbN control culture. This suggests that the vandefitemcel (or SB623 cells) promoted more glial differentiation in the forebrain neurons.

### Example 2: Coculturing Vandefitemcel With iFbN Cells Results in More Neuronal Activity Than Coculturing iFbN Cells With Astrocytes and Molecules Secreted by Vandefitemcel Increase Neuronal Activity

Cocultures of vandefitemcel (or SB623 cells) and iFbNs were compared to cocultures of human astrocytes (hA) and iFbNs to investigate whether the increased effect on neuronal activity was caused by extra cells in the MEA system. Also, iFbNs were cultured with conditioned medium (CM) of vandefitemcel to evaluate whether secreted factors could have a similar effect on neuronal activity. Conditioned medium was collected every day from vandefitemcel (or SB623 cells) cultured in BrainPhys™ medium and immediately added to cultures of iFbN cells when medium was replaced. iFbN cells were cultured for two weeks before other cells were added; at two weeks, vandefitemcel, conditioned medium of vandefitemcel, and human astrocytes were added and spikes and network bursts were counted in each case.

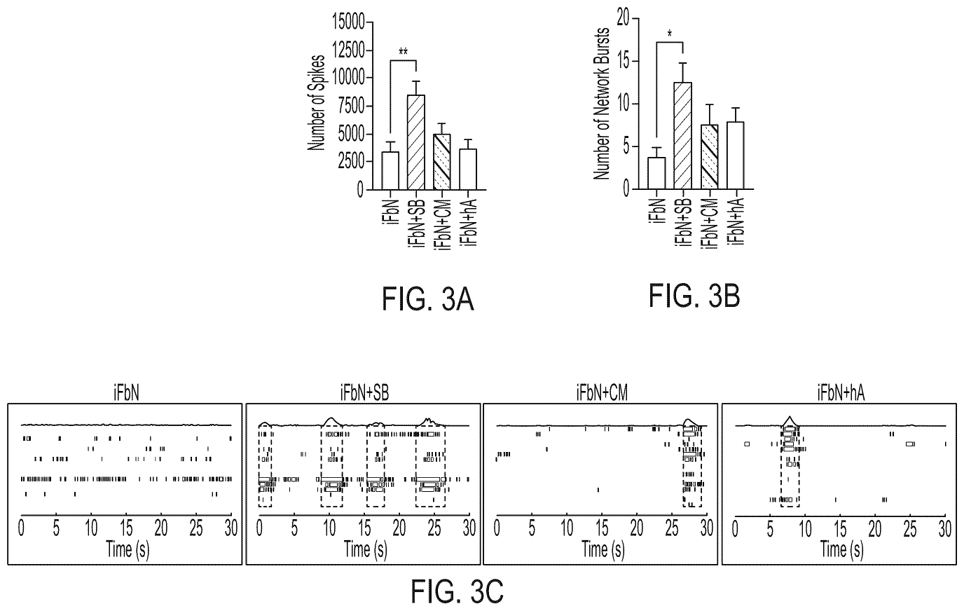

As shown in FIGS. 3A-3C, in the subsequent two weeks after vandefitemcel was added to the iFbN cells, the coculture of vandefitemcel and iFbN (shown in FIGS. 3A-3C as “iFbN+SB”) generated significantly more spikes (see FIG. 3A, one-way ANOVA Holm-Sidak, **p<0.01) and network bursts (see FIG. 3B, one-way ANOVA Holm-Sidak, *p<0.05, and FIG. 3C) than the iFbN cells alone (shown in FIGS. 3A-3C as “iFbN”). Moreover, it can be seen in FIGS. 3A-3C that cocultures of conditioned medium of vandefitemcel and iFbN (shown in FIGS. 3A-3C as “iFbN+CM”) also generated more spikes and network bursts than the iFbN cells alone (although not to the same extent as the iFbN+SB). Furthermore, cocultures of human astrocytes and iFbN (shown in FIGS. 3A-3C as “iFbN+hA”) also generated more spikes and network bursts than the iFbN cells alone (although not to the same extent as the iFbN+SB).

FIG. 3C are raster plots illustrating the electrophysiological activity of cultures of iFbN alone and cocultures of iFbN with vandefitemcel, conditioned medium (CM) of vandefitemcel, and human astrocytes (hA) at four weeks of culture across electrodes of the MEA system. Single spikes are represented in the plots by black boxes, electrode bursts are represented in the plots by white boxes, and network bursts are represented by broken-line boxes. Altogether, these results suggest that molecules secreted from vandefitemcel resulted in an increase in neuronal activity but the presence of vandefitemcel cells in culture significantly augmented this effect.

### Example 3: Coculturing Vandefitemcel With Glutamatergic Neurons Results in More Neuronal Activity

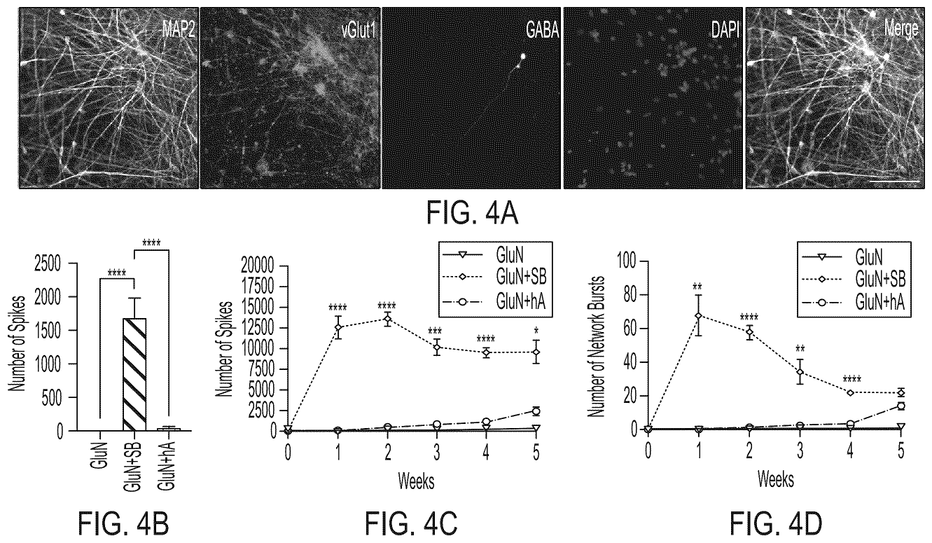

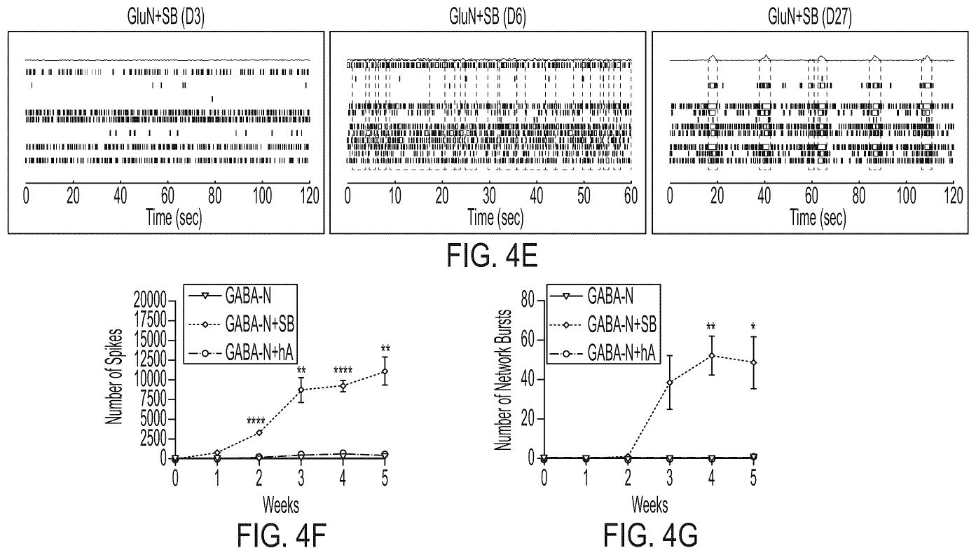

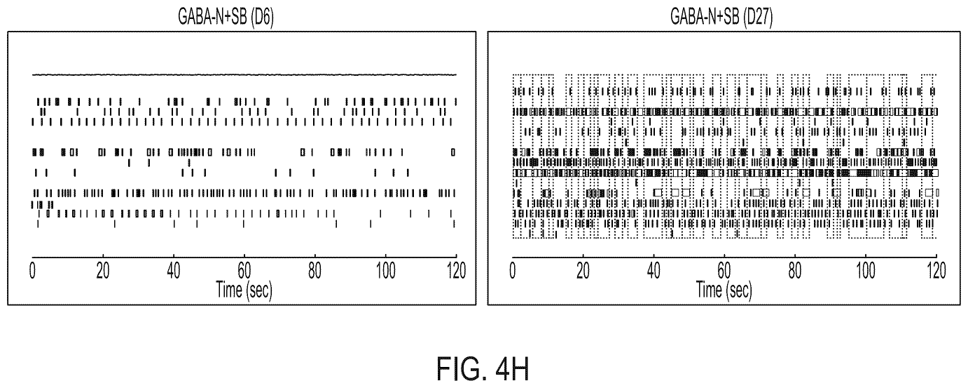

Neuronal network bursts, or neuronal oscillations, are generated by intrinsic neuronal connections between excitatory and inhibitory neurons. FIG. 4A are immunocytochemistry (ICC) stains of a culture of iFbNs, at thirty days of culture maturation, showing the neurons expressing the neuronal marker MAP2 as well as excitatory vesicular glutamate transporter (vGLUT1) and inhibitory y-aminobutyric acid (GABA). Cell nuclei were stained with DAPI (scale: 50 μm). The ICC stains showed abundant expression of vGLUT1, indicating the presence of excitatory neurons. This observation was confirmed by a reduction in the spike activity when kynurenic acid (50 μM), a broad glutamate receptor antagonist, was added to the iFbN culture.

To investigate the effects of vandefitemcel (or SB623) cells on an excitatory neuronal population, vandefitemcel was cocultured with an enriched population of iPSC-derived glutamatergic neurons (hereinafter, “glutamatergic neurons”). Unlike the iFbNs previously used in these experiments, the glutamatergic neurons were mature at the first day of culture, and did not need two weeks of maturation. Therefore, glutamatergic neurons and vandefitemcel were cocultured together on day 1. The effect of vandefitemcel on glutamatergic neurons (shown in FIGS. 4B-4E as “GluN+SB”) was compared to that of glutamatergic neurons alone (shown in FIGS. 4B-4D as “GluN”) or glutamatergic neurons in coculture with human astrocytes (shown in FIGS. 4B-4D as “GluN+hA”) at the same cell density as that of vandefitemcel (or SB623 cells).

FIG. 4B illustrates that on day three of coculture, vandefitemcel promoted high levels of spike activity in glutamatergic neurons, when compared to spike activity in either the glutamatergic neurons alone or the glutamatergic neurons and human astrocytes cocultures (one-way ANOVA Holm-Sidak, ****p<0.0001). FIG. 4C illustrates that this spike activity kept increasing for two weeks, while human astrocytes (hA) slightly promoted spike activity in coculture with glutamatergic neurons starting at week three (two-way ANOVA Tukey, *p<0.05; ***p<0.001; ****p<0.0001). FIG. 4D illustrates that vandefitemcel (or SB623 cells) induced more network burst activity in the first week of coculture with glutamatergic neurons (GluN+SB), while glutamatergic neurons in coculture with human astrocytes (GluN+hA) did not show network bursts until week four (two-way ANOVA Tukey, *p<0.05; **; <0.005; ***p<0.001; ****p<0.0001). FIGS. 4D and 4E also illustrate that network bursts in cocultures of glutamatergic neurons with vandefitemcel (GluN+SB) reduced after the first week, indicating the presence of an inhibitory input in this culture. FIGS. 4C and 4D illustrate that a culture of glutamatergic neurons alone (GluN) had lower spike activity compared to cocultures with either human astrocytes (GluN+hA) or vandefitemcel (GluN+SB) and failed to form network bursts for five weeks of culture.

FIG. 4E are raster plots illustrating the electrophysiological activity of the GluN+SB coculture and progressive network burst development across electrodes on the MEA system. On day three (D3), there were many spikes observed in the GluN+SB coculture. On day six (D6), many spikes and network bursts were observed in the GluN+SB coculture. On day 27 (D27), there was a drop in spike counts and network burst counts, indicating the input of inhibitory synapses between network bursts. Single spikes are represented in the plots by black boxes, electrode bursts are represented in the plots by white boxes, and network bursts arc represented by broken-line boxes.

To investigate the effects of vandefitemcel (or SB623) cells on GABAergic neurons, the same experiments were repeated using an enriched population of iPSC-derived GABAergic neurons (hereinafter, “GABAergic neurons”). As shown in FIGS. 4F and 4G, vandefitemcel (or SB623 cells) promoted spikes and network bursts of GABAergic neurons in week two and week four of coculture when compared to either in GABAergic neurons alone (shown as “GABA-N”) or in cocultures of GABAergic neurons and human astrocytes (shown as “GABA-N+hA”) (two-way ANOVA Tukey, *p<0.05; **p<0.005; ****p<0.0001). While the number of network bursts induced by vandefitemcel in coculture with GABAergic neurons reached a similar level to that observed in coculture with glutamatergic neurons, it took longer to reach this peak (sec FIGS. 4E and 4H). Like glutamatergic neurons, GABAergic neurons alone did not exhibit network bursts, indicating that supporting cells are required to promote electrical activity in GABAergic neurons. Interestingly, a decrease in resistance at week one of culture was observed, followed by recovery at week two, and then a continuous decline until week five. This pattern mirrors the observations in vandefitemcel (or SB623 cells) cultured alone (see FIG. 1D). These findings indicate that vandefitemcel (or SB623 cells) induced an earlier, and higher, spike activity in enriched glutamatergic neurons, suggesting that SB623 cells play a role in modulating the synaptic activity of excitatory neurons.

FIG. 4H are raster plots illustrating the electrophysiological activity of the GABA-N+SB coculture and progressive network burst development across electrodes on the MEA system. There was no spike activity on day three of the GABA-N+SB623 coculture (not shown). On day six (D6), many spikes were observed in the GABA-N+SB coculture. On day 27 (D27), many spikes and network bursts were observed in the GABA-N+SB coculture. Single spikes are represented in the plots by black boxes, electrode bursts are represented in the plots by white boxes, and network bursts are represented by broken-line boxes.

### Example 4: Vandefitemcel Increases Neuronal Activity and Promotes Synaptic Plasticity by Tonic Glutamate Release

Glutamate, the main excitatory neurotransmitter in the mammalian nervous system, is involved in synapse formation [24]. We first looked at the amount of glutamate released by vandefitemcel by comparing the levels of glutamate in conditioned medium (CM) of vandefitemcel (or SB623 cells) with that in the CM of human astrocytes (hA). Vandefitemcel (or SB623 cells) and human astrocytes were cultured in BrainPhys™ medium for five days at different cell densities.

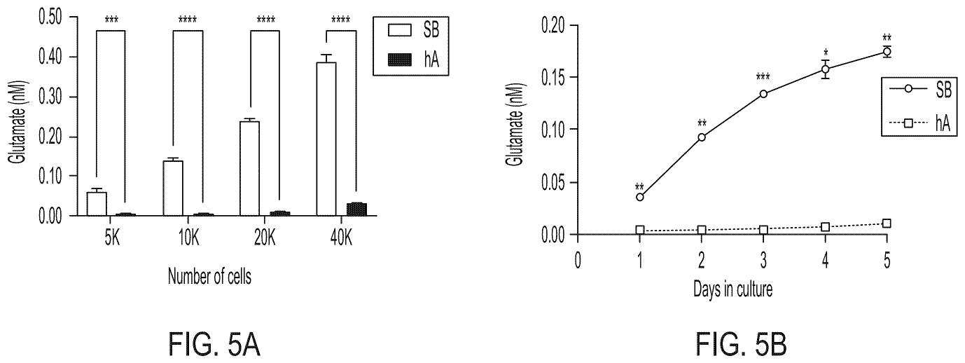

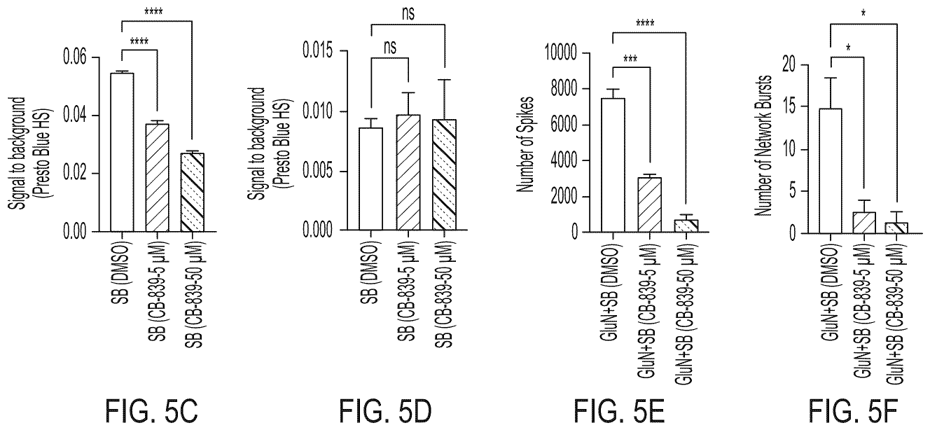

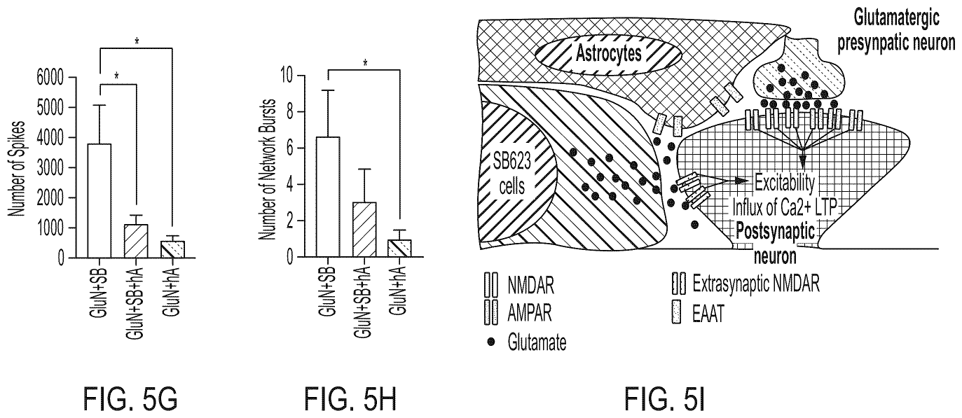

FIG. 5A shows that at two days of culture, the increased levels of CM glutamate were cell density-dependent in cultures of both cell types, but the levels of glutamate in CM of vandefitemcel (or SB623 cells, shown as “SB” in FIG. 5A) were about 10-fold higher than that in the CM of human astrocytes (shown as “hA” in FIG. 5A) (two-way ANOVA Holm-Sidak, ***p<0.001; ****p<0.0001). FIG. 5B shows that at five days of culture, the levels of glutamate in CM of vandefitemcel (or SB623 cells, shown as “SB” in FIG. 5B) increased 4.9-fold, while the levels of glutamate in CM of human astrocytes (shown as “hA” in FIG. 5B) increased 2.7-fold, indicating that tonic glutamate release by vandefitemcel was higher than that in human astrocytes (two-way ANOVA Holm-Sidak, *p<0.05; **p<0.005; ***p<0.001).

Glutamate is the catalysis of glutamine by glutaminase. To explore whether glutaminase plays a role in the glutamate release by vandefitemcel (or SB623 cells), glutamine was added to the BrainPhys™ medium and the levels of glutamate in the CM were assessed. At two days of culture, the levels of glutamate in the CM increased 1.6-fold suggesting that glutaminase indeed played a role in the glutamate released by vandefitemcel.

Next, experiments were performed to determine if inhibition of glutaminase in vandefitemcel (or SB623 cells) would affect the excitability observed in the coculture of vandefitemcel and glutamatergic neurons (GluN). To this end, the activity of glutaminase was inhibited in vandefitemcel with Telaglenastat (CB-839). Vandefitemcel (or SB623 cells) were incubated for two days with CB-839 and maintained in BrainPhys™ medium for three days to assess the levels of glutamate in the CM.

FIG. 5C illustrates that after incubation with 5 μM and 50 μM of CB-839, the levels of glutamate in the CM of vandefitemcel (shown as “SB (CB-839-5 μM)” and “SB (CB-839-50 μM)” in FIG. 5C) were reduced 1.5-fold and 2.0-fold, respectively, compared to the control of vandefitemcel incubated with dimethylsulfoxide (DMSO) (shown as “SB (DMSO)” in FIG. 5C) (one-way ANOVA Holm-Sidak, ****p<0.0001).

Glutamate can be involved in cell metabolism, where it is transformed into α-ketoglutarate and then enters into the tricarboxylic acid cycle to generate ATP [25]. Therefore, to evaluate whether inhibition of glutaminase would affect cell metabolism and viability, the viability of vandefitemcel (or SB623 cells) treated with CB-839 was assessed using PrestoBlue™ high-sensitivity (HS) Cell Viability Reagent (from Thermo Fisher Scientific, Waltham, MA). FIG. 5D illustrates that incubation with CB-839 did not alter the viability of vandefitemcel (or SB623 cells) (one-way ANOVA Holm-Sidak, “ns” =no difference in cell viability).

Next, glutamatergic neurons were cocultured with vandefitemcel that were pre-incubated with both 5 μM and 50 μM of CB-839 to inhibit glutaminase. FIGS. 5E and 5F show that after one week of coculture, the inhibition of glutaminase in vandefitemcel significantly reduced the spike activity (see FIG. 5E) and network bursts (see FIG. 5F) in the cocultures of glutamatergic neurons and vandefitemcel (shown as “GluN+SB (CB-839—5 μM)” and “GluN+SB (CB-839—50 μM)” compared to the control culture of vandefitemcel incubated with dimethylsulfoxide (DMSO) (shown as “SB (DMSO)” in FIGS. 5E and 5F) (one-way ANOVA Holm-Sidak, ***p<0.0005; ****p<0.0001).

Lastly, experiments were performed to investigate whether human astrocytes (hA) could diminish the effect of vandefitemcel on glutamatergic neurons. Astrocytes have a key role in brain homeostasis by the uptake of glutamate from the extracellular space [26]. Thus, three cocultures were prepared including 1) glutamatergic neurons with vandefitemcel (shown in FIGS. 5G and 5H as “GluN+SB”), 2) glutamatergic neurons with vandefitemcel and human astrocytes (shown in FIGS. 5G and 5H as “GluN+SB+hA”), and 3) glutamatergic neurons with human astrocytes (shown in FIGS. 5G and 5H as “GluN+hA”). Neuronal activity and synaptic plasticity were assessed in the form of the number of spikes (i.e., action potentials that appear in the signal when neurons fire) and the number of network bursts.

FIGS. 5G and 5H illustrate that astrocytes did indeed reduce the number of spikes and network bursts that would have been otherwise induced by vandefitemcel (or SB623 cells) on the glutamatergic neurons (one-way ANOVA Holm-Sidak, *p<0.05). This data demonstrates that the increased neuronal activity and synaptic plasticity driven by vandefitemcel was caused by tonic glutamate release.

FIG. 5I is a schematic diagram showing vandefitemcel (or SB623 cells) promoting an increase in neuronal activity and synaptic plasticity via tonic glutamate release.

The results of the experiments disclosed herein show that vandefitemcel (or SB623 cells), which are genetically modified bone marrow-derived MSCs, increase neuronal activity and synaptic plasticity in human neurons, and this effect is induced through tonic glutamate release by vandefitemcel. As will be discussed in the following sections, the findings from these experiments can be applied to MSC-based cell therapies involving the transplantation of vandefitemcel (or SB623 cells) in human and animal subjects.

Moreover, functional magnetic resonance imaging (fMRI) [27] can be used prior to cell transplantation to help determine optimal target brain regions for transplantation. For example, fMRI can be used to select region(s) of the brain showing neuronal activity and vandefitemcel can be administered to such region(s) of the brain selected based on the fMRI scans. This can result in better efficacy of cell transplantation procedures and reduction in clinical outcome variability and treatment-emergent adverse events.

### Methods of Treatment

One unexpected result from the experiments described herein is that the introduction of vandefitemcel to neurons induces, causes, or triggers tonic release of glutamate to such neurons. Another unexpected result from the experiments described herein is that the introduction of vandefitemcel to neurons increases synapse formation in such neurons.

Based on the results of the experiments disclosed herein, a method of inducing, causing, or triggering the tonic release of glutamate in a subject comprises administering vandefitemcel to a region of the brain of the subject, wherein the vandefitemcel are cells descended from mesenchymal stem cells (MSCs) transiently-transfected by a polynucleotide encoding a Notch intracellular domain (NICD).

Administering the vandefitemcel can further comprise administering the vandefitemcel by intracerebral implantation. The vandefitemcel can tonically release glutamate to neurons of the brain when implanted via intracerebral implantation.

Administering the vandefitemcel can further comprise injecting the vandefitemcel at multiple sites within the brain of the subject. In some cases, the vandefitemcel can be administered stereotactically via a burr hole in the skull of a subject.

Additional details on stereotactic administration of the cells can be found in U.S. Pat. No. 11,439,761, the content of which is incorporated herein by reference in its entirety for the purposes of describing stereotactic administration of vandefitemcel and equipment used for such purposes.

Administering the vandefitemcel can also comprise administering the vandefitemcel to a site within a forebrain of the subject including at least one of the cerebrum, the thalamus, and the hypothalamus of the subject.

Administering the vandefitemcel can further comprise administering the vandefitemcel to a hippocampus of the subject.

Administering the vandefitemcel can further comprise administering the vandefitemcel to a site of injury or disease in the brain region of the subject.

Administering the vandefitemcel can comprise administering the vandefitemcel by parenteral administration.

The vandefitemcel can be suspended in a sterile isotonic crystalloid solution. For example, the cell suspension can comprise the cells suspended in Plasma-Lyte™ A (Baxter Healthcare Corporation). The cells can also be suspended in another physiologically compatible carrier such as phosphate buffered saline.

The therapeutically effective amount of vandefitemcel can be between about 1.0million cells and 10.0 million cells (e.g., about 1.0 million cells, 1.5 million cells, 2.0 million cells, 2.5 million cells, 3.0 million cells, 3.5 million cells, 4.0 million cells, 4.5 million cells, 5.0 million cells, 5.5 million cells, 6.0 million cells, 6.5 million cells, 7.0 million cells, 7.5 million cells, 8.0 million cells, 8.5 million cells, 9.0 million cells, 9.5 million cells, 10.0 million cells, and amounts therebetween).

As disclosed herein, the vandefitemcel can be made by a method comprising: (i) providing a culture of mesenchymal stem cells; (ii) contacting the culture of mesenchymal stem cells with a polynucleotide encoding a Notch intracellular domain (NICD) and where the polynucleotide does not encode a full-length Notch protein; (iii) selecting cells that comprise the polynucleotide; and (iv) further culturing the selected cells in the absence of selection for the polynucleotide. The mesenchymal stem cells can be human bone marrow-derived cells.

As disclosed herein, the method can further comprise capturing functional magnetic resonance imaging (fMRI) scans of regions of the brain of the subject, selecting one region of the brain showing neuronal activity based on the fMRI scans, and administering the vandefitemcel to the one region of the brain.

As disclosed herein, the vandefitemcel can be made by a method comprising: (i) providing a culture of mesenchymal stem cells; (ii) contacting the culture of mesenchymal stem cells with a polynucleotide encoding a Notch intracellular domain (NICD) and where the polynucleotide does not encode a full-length Notch protein; (iii) selecting cells that comprise the polynucleotide; and (iv) further culturing the selected cells in the absence of selection for the polynucleotide. The mesenchymal stem cells can be human bone marrow-derived cells.

As disclosed herein, the method can further comprise capturing functional magnetic resonance imaging (fMRI) scans of regions of the brain of the subject, selecting one region of the brain showing neuronal activity based on the fMRI scans; and administering the vandefitemcel to the one region of the brain.

Also disclosed is a method of increasing synapse formation in neurons, comprising administering vandefitemcel to a region of the brain of the subject comprising the neurons, wherein the vandefitemcel are cells descended from mesenchymal stem cells (MSCs) transiently-transfected by a polynucleotide encoding a Notch intracellular domain (NICD).

Administering the vandefitemcel can further comprise administering the vandefitemcel by intracerebral implantation. The vandefitemcel can tonically release glutamate to neurons of the brain when implanted via intracerebral implantation.

Administering the vandefitemcel further comprises injecting the vandefitemcel at multiple sites within the brain of the subject. In some cases, the vandefitemcel can be administered stereotactically via a burr hole in the skull of a subject.

Administering the vandefitemcel can further comprise administering the vandefitemcel to a hippocampus of the subject.

Administering the vandefitemcel can further comprise administering the vandefitemcel to a site of injury or disease in the brain region of the subject.

Administering the vandefitemcel can comprise administering the vandefitemcel by parenteral administration.

The vandefitemcel can be suspended in a sterile isotonic crystalloid solution. For example, the cell suspension can comprise the cells suspended in Plasma-Lyte™ A (Baxter Healthcare Corporation). The cells can also be suspended in another physiologically compatible carrier such as phosphate buffered saline.

The therapeutically effective amount of vandefitemcel can be between about 1.0million cells and 10.0 million cells (e.g., about 1.0 million cells, 1.5 million cells, 2.0 million cells, 2.5 million cells, 3.0 million cells, 3.5 million cells, 4.0 million cells, 4.5 million cells, 5.0 million cells, 5.5 million cells, 6.0 million cells, 6.5 million cells, 7.0 million cells, 7.5 million cells, 8.0 million cells, 8.5 million cells, 9.0 million cells, 9.5 million cells, 10.0 million cells, and amounts therebetween).

As disclosed herein, the vandefitemcel can be made by a method comprising: (i) providing a culture of mesenchymal stem cells; (ii) contacting the culture of mesenchymal stem cells with a polynucleotide encoding a Notch intracellular domain (NICD) and where the polynucleotide does not encode a full-length Notch protein; (iii) selecting cells that comprise the polynucleotide; and (iv) further culturing the selected cells in the absence of selection for the polynucleotide. The mesenchymal stem cells can be human bone marrow-derived cells.

As disclosed herein, the method can further comprise capturing functional magnetic resonance imaging (fMRI) scans of regions of the brain of the subject, selecting one region of the brain showing neuronal activity based on the fMRI scans; and administering the vandefitemcel to the one region of the brain.

Also disclosed is a method of inducing, causing, or triggering the release of glutamate in a subject. The method can comprise capturing functional magnetic resonance imaging (fMRI) scans of regions of the brain of the subject, selecting a region of the brain showing neuronal activity based on the fMRI scans, and administering vandefitemcel to the region of the brain selected. The vandefitemcel are cells descended from mesenchymal stem cells (MSCs) transiently-transfected by a polynucleotide encoding a Notch intracellular domain (NICD). The vandefitemcel can release glutamate after being administered via intracerebral implantation.

Also disclosed is a method for increasing synapse formation in neurons. The method can comprise capturing functional magnetic resonance imaging (fMRI) scans of regions of the brain of the subject, selecting a region of the brain showing neuronal activity based on the fMRI scans, and administering vandefitemcel to the region of the brain selected comprising the neurons. The vandefitemcel are cells descended from mesenchymal stem cells (MSCs) transiently-transfected by a polynucleotide encoding a Notch intracellular domain (NICD). The vandefitemcel can release glutamate to the neurons after being administered via intracerebral implantation.

### Composition, Formulations, and Kits

The composition can increase synapse formation in neurons and can comprise vandefitemcel in an amount between about 1.0 million cells and 10.0 million cells. The vandefitemcel can be produced by modifying mesenchymal stem cells derived from bone marrow. The composition can also comprise one or more pharmaceutically acceptable excipients.

As previously discussed, the vandefitemcel can be descended from mesenchymal stem cells transiently-transfected by a polynucleotide encoding a Notch intracellular domain (NICD). The vandefitemcel can be made by a process comprising providing a culture of the mesenchymal stem cells (i.e., human bone marrow-derived cells) and contacting the culture of mesenchymal stem cells with the polynucleotide encoding the NICD. In certain instances, the polynucleotide does not encode a full-length Notch protein. The vandefitemcel can further be made by selecting cells that comprise the polynucleotide; and further culturing the selected cells in the absence of selection for the polynucleotide.

The composition can further comprise additional mesenchymal stem cells not transiently-transfected by the polynucleotide.

The mesenchymal stem cells can be transiently-transfected with a plasmid vector comprising the polynucleotide encoding the NICD.

The vandefitemcel can be suspended in a sterile isotonic crystalloid solution.

The one or more pharmaceutically acceptable excipients can comprise at least one of buffers, proteins, stabilizers, and preservatives. The one or more pharmaceutically acceptable excipients can also comprise carriers or diluents to form the cell suspension.

The pharmaceutically acceptable carrier can be a physiologically compatible carrier for implantation. As used herein, the term “physiologically compatible carrier” can refer to a carrier that is compatible with the other ingredients of the formulation and not deleterious to the recipient thereof. Examples of suitable carriers or diluents include cell culture medium (e.g., Eagle's minimal essential medium), phosphate buffered saline, Hank's balanced salt solution+/−glucose (HBSS), and multiple electrolyte solutions. The pharmaceutically acceptable carrier or diluent can also be or comprise a sterile isotonic crystalloid solution such as Plasma-Lyte™ A (Baxter Healthcare Corporation).

The composition can comprise the vandefitemcel packaged in a sealed vial. In some instances, the sealed vial can comprise 0.3 mL of the cell suspension with a cell concentration of approximately 8.5*10⁶ cells/mL. Alternatively, the sealed vial can comprise 0.3 mL of the cell suspension with a cell concentration of approximately 17.0*10⁶ cells/mL.

Also disclosed are other examples of materials which can serve as pharmaceutically-acceptable carriers or excipients including: sugars, such as lactose, glucose and sucrose; starches, such as corn starch and potato starch; cellulose and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-frec water; isotonic saline; Ringer's solution; ethyl alcohol; phosphate buffer solutions; and other non-toxic compatible substances. Wetting agents, emulsifiers and lubricants, such as sodium lauryl sulfate and magnesium stearate, as well as coloring agents, release agents, coating agents, sweetening, flavoring and perfuming agents, preservatives, and antioxidants can also be present in the composition.

Other examples of pharmaceutically-acceptable carriers or excipients include substances that stimulate angiogenesis (“pro-angiogenic agents”). In some instances, the pro-angiogenic agent can be a protein (e.g., fibroblast growth factor, platelet-derived growth factor, transforming growth factor alpha, hepatocyte growth factor, vascular endothelial growth factor, sonic hedgehog, MAGP-2, HIF-1, PR-39, RTEF-1, c-Myc, TFII, Egr-1, ETS-1) or a nucleic acid encoding such a protein. See, for example, Vincent et al. (2007) Gene Therapy 14:781-789. In other instances, the pro-angiogenic agent can be a small RNA molecule (e.g., siRNA, shRNA, microRNA) or a ribozyme that targets a nucleic acid encoding an inhibitor of angiogenesis. Moreover, the pro-angiogenic agent can be a triplex-forming nucleic acid that binds to DNA sequences regulating the expression of a protein that inhibits angiogenesis, so as to block transcription of the gene encoding the protein.

Exemplary formulations include, but are not limited to, those suitable for parenteral administration, e.g., intrapulmonary, intra-arterial, intra-ocular, intra-cranial, sub-meningeal, or subcutaneous administration, including formulations encapsulated in micelles, liposomes or drug-release capsules (active agents incorporated within a biocompatible coating designed for slow-release); ingestible formulations; formulations for topical use, such as eye drops, creams, ointments and gels; and other formulations such as inhalants, aerosols and sprays. The dosage of the compositions of the disclosure can vary according to the extent and severity of the need for treatment, the activity of the administered composition, the general health of the subject, and other considerations well known to the skilled artisan.

In additional embodiments, the compositions described herein can also be delivered locally. Localized delivery allows for the delivery of the composition non-systemically, thereby reducing the body burden of the composition as compared to systemic delivery. Such local delivery can be achieved, for example, through the use of various medically implanted devices including, but not limited to, stents and catheters, or can be achieved by inhalation, injection or surgery. Methods for coating, implanting, embedding, and otherwise attaching desired agents to medical devices such as stents and catheters are established in the art and contemplated herein.

Another aspect of the present disclosure relates to kits for carrying out the administration of the cells to a subject. For example, the kit can comprise the composition of cells, formulated as appropriate (e.g., in a pharmaceutical carrier), in one or more separate pharmaceutical preparations.

Compositions comprising vandefitemcel can be used in combination with other compositions comprising substances that stimulate angiogenesis (“pro-angiogenic agents”). The compositions can be administered sequentially in any order or concurrently. Accordingly, therapeutic compositions as disclosed herein can contain both vandefitemcel and a pro-angiogenic agent. In additional embodiments, separate therapeutic compositions, one comprising vandefitemcel and the other comprising a pro-angiogenic agent, can be administered to the subject, either separately or together.

The pro-angiogenic agent can be a transcription factor that activates expression of a pro-angiogenic molecule (e.g., protein). Naturally-occurring transcription factors (such as, for example, HIF-1alpha) that regulate the expression of pro-angiogenic proteins, are known. In addition, synthetic transcriptional regulatory proteins can be constructed by genetic engineering. For example, methods for the design of zinc finger DNA-binding domains that bind to a sequence of interest, and methods for the fusion of such zinc finger DNA-binding domains to transcriptional activation and repression domains, have been described. See, for example, U.S. Pat. Nos. 6,534,261; 6,607,882; 6,785,613; 6,794,136; 6,824,978; 6,979,539; 7,013,219; 7,177,766; 7,220,719; and 7,788,044. These methods can be used to synthesize non-naturally-occurring proteins that activate transcription of any gene encoding a pro-angiogenic protein. In addition, synthetic zinc finger transcriptional activators of the vascular endothelial growth factor (VEGF) gene have been described. See, e.g., U.S. Pat. Nos. 7,026,462; 7,067,317; 7,560,440; 7,605,140; and 8,071,564. Accordingly, a non-naturally-occurring (i.e., synthetic) zinc finger protein that activates transcription of the VEGF gene can be used, in combination with vandefitemcel, for augmenting angiogenesis, e.g., in the treatment of stroke. Furthermore, a natural or synthetic transcriptional regulatory protein (e.g., a synthetic zinc finger transcriptional regulatory protein) that inhibits transcription of an anti-angiogenic molecule can also be used as a pro-angiogenic agent.

A number of embodiments have been described. Nevertheless, it will be understood by one of ordinary skill in the art that various changes and modifications can be made to this disclosure without departing from the spirit and scope of the embodiments. Elements of systems, devices, apparatus, and methods shown with any embodiment are exemplary for the specific embodiment and can be used in combination or otherwise on other embodiments within this disclosure. For example, the steps of any methods depicted in the figures or described in this disclosure do not require the particular order or sequential order shown or described to achieve the desired results. In addition, other steps or operations may be provided, or steps or operations may be eliminated or omitted from the described methods or processes to achieve the desired results. Moreover, any components or parts of any apparatus or systems described in this disclosure or depicted in the figures may be removed, eliminated, or omitted to achieve the desired results. In addition, certain components or parts of the systems, devices, or apparatus shown or described herein have been omitted for the sake of succinctness and clarity.

Accordingly, other embodiments are within the scope of the following claims and the specification and/or drawings may be regarded in an illustrative rather than a restrictive sense.

Each of the individual variations or embodiments described and illustrated herein has discrete components and features which may be readily separated from or combined with the features of any of the other variations or embodiments. Modifications may be made to adapt a particular situation, material, composition of matter, process, process act(s) or step(s) to the objective(s), spirit or scope of the present invention.

Methods recited herein may be carried out in any order of the recited events that is logically possible, as well as the recited order of events. Moreover, additional steps or operations may be provided or steps or operations may be eliminated to achieve the desired result.

Furthermore, where a range of values is provided, every intervening value between the upper and lower limit of that range and any other stated or intervening value in that stated range is encompassed within the invention. Also, any optional feature of the inventive variations described may be set forth and claimed independently, or in combination with any one or more of the features described herein. For example, a description of a range from 1 to 5 should be considered to have disclosed subranges such as from 1 to 3, from 1 to 4, from 2 to 4, from 2 to 5, from 3 to 5, etc. as well as individual numbers within that range, for example 1.5, 2.5, etc. and any whole or partial increments therebetween.

All existing subject matter mentioned herein (e.g., publications, patents, patent applications) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art to which this invention belongs.

Reference to the phrase “at least one of,” when such phrase modifies a plurality of items or components (or an enumerated list of items or components) means any combination of one or more of those items or components. For example, the phrase “at least one of A, B, and C” means: (i) A; (ii) B; (iii) C; (iv) A, B, and C; (v) A and B; (vi) B and C; or (vii) A and C.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including,” “having,” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved.

Finally, terms of degree such as “substantially”, “about” and “approximately” as used herein mean the specified value or the specified value and a reasonable amount of deviation from the specified value (e.g., a deviation of up to ±0.1%, ±1%, ±5%, or ±10%, as such variations are appropriate) such that the end result is not significantly or materially changed. For example, “about 1.0 cm” can be interpreted to mean “1.0 cm” or between “0.9 cm and 1.1 cm.” When terms of degree such as “about” or “approximately” are used to refer to numbers or values that are part of a range, the term can be used to modify both the minimum and maximum numbers or values.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that may become obvious to those skilled in the art in view of this disclosure.

### REFERENCES

1. He, J. Q., Sussman, E. S., Steinberg, G. K., 2020. Revisiting Stem Cell-Based Clinical Trials for Ischemic Stroke. Front Aging Neurosci 12, 575990.
2. Volkman, R., Offen, D., 2017. Concise Review: Mesenchymal Stem Cells in Neurodegenerative Diseases. Stem Cells 35, 1867-1880.
3. Berebichez-Fridman, R., Montero-Olvera, P. R., 2018. Sources and Clinical Applications of Mesenchymal Stem Cells: State-of-the-art review. Sultan Qaboos Univ Med J 18, e264-e277.
4. Kawabori, M., Weintraub, A. H., Imai, H., Zinkevych, L., McAllister, P., Steinberg, G. K., Frishberg, B. M., Yasuhara, T., Chen, J. W., Cramer, S. C., Achrol, A. S., Schwartz, N. E., Suenaga, J., Lu, D. C., Semeniv, I., Nakamura, H., Kondziolka, D., Chida, D., Kaneko, T., Karasawa, Y., Paadre, S., Nejadnik, B., Bates, D., Stonehouse, A. H., Richardson, R. M., Okonkwo, D. O., 2021. Cell Therapy for Chronic TBI: Interim Analysis of the Randomized Controlled STEMTRA Trial. Neurology 96, e1202-1214.
5. Steinberg, G. K., Kondziolka, D., Wechsler, L. R., Lunsford, L. D., Kim, A. S., Johnson, J. N., Bates, D., Poggio, G., Case, C., McGrogan, M. and Yankee, E. W., 2018. Two-year safety and clinical outcomes in chronic ischemic stroke patients after implantation of modified bone marrow-derived mesenchymal stem cells (SB623): a phase ½a study. Journal of Neurosurgery, 131(5), pp.1462-1472.
6. Wang, S., Hesen, R., Mossink, B., Kasri, N. N. and Schubert, D., 2023. Generation of glutamatergic/GABAergic neuronal co-cultures derived from human induced pluripotent stem cells for characterizing E/I balance in vitro. STAR protocols, 4(1), p.101967.
7. De Filippi, G., Hayes, H. B., Nicolini, A. M., Arrowood, C. A. and Millard, D. C., 2020. Multiwell Microelectrode Array (MEA) Technology for the quantification of neuronal, synaptic, and network function for in vitro primary and stem cell-derived neuronal models. Journal of Pharmacological and Toxicological Methods, 105, p.106769.
8. Jiang, Yuehua, et al. Pluripotency of mesenchymal stem cells derived from adult marrow. Nature 418.6893 (2002): 41-49.
9. Pittenger, Mark F., et al. Multilineage potential of adult human mesenchymal stem cells. Science 284.5411 (1999): 143-147.
10. Dezawa, Mari, et al. Sciatic nerve regeneration in rats induced by transplantation of in vitro differentiated bone-marrow stromal cells. European Journal of Neuroscience 14.11 (2001): 1771-1776.
11. Campagnoli, Cesare, et al. Identification of mesenchymal stem/progenitor cells in human first-trimester fetal blood, liver, and bone marrow. Blood, The Journal of the American Society of Hematology 98.8 (2001): 2396-2402.
12. Erices, Alejandro, Paulette Conget, and José J. Minguell. Mesenchymal progenitor cells in human umbilical cord blood. British journal of haematology 109.1 (2000): 235-242.
13. Artavanis-Tsakonas, Spyros, Kenji Matsuno, and Mark E. Fortini. Notch signaling. Science 268.5208 (1995): 225-232.
14. Mumm, Jeffrey S., and Raphael Kopan. Notch signaling: from the outside in. Developmental biology 228.2 (2000): 151-165.
15. Ehebauer, Matthias, Penelope Hayward, and Alfonso Martinez-Arias. Notch signaling pathway. Science's STKE 2006.364 (2006): cm7-cm7.
16. Weinmaster, Gerry, Veronica J. Roberts, and Greg Lemke. A homolog of Drosophila Notch expressed during mammalian development. Development 113.1 (1991): 199-205.
17. Schroeter, Eric H., Jeffrey A. Kisslinger, and Raphael Kopan. Notch-1 signalling requires ligand-induced proteolytic release of intracellular domain. Nature 393.6683 (1998): 382-386.
18. Dezawa, Mari, et al. Specific induction of neuronal cells from bone marrow stromal cells and application for autologous transplantation. The Journal of clinical investigation 113.12 (2004): 1701-1710.
19. Sambrook, Joseph, Edward F. Fritsch, and Tom Maniatis. Molecular cloning: a laboratory manual. No. Ed. 2. Cold spring harbor laboratory press, 1989.
20. Brent, R., et al. Current Protocols in Molecular Biology (Janssen, K., ed) Vol. 1. (1994).
21. Bardy, C., van den Hurk, M., Eames, T., Marchand, C., Hernandez, R. V., Kellogg, M., Gorris, M., Galet, B., Palomares, V., Brown, J., Bang, A. G., Mertens, J., Bohnke, L., Boyer, L., Simon, S., Gage, F. H., 2015. Neuronal medium that supports basic synaptic functions and activity of human neurons in vitro. Proc Natl Acad Sci U S A 112, E2725-2734.
22. Aizman, I., Tate, C. C., McGrogan, M., Case, C. C., 2009. Extracellular matrix produced by bone marrow stromal cells and by their derivative, SB623 cells, supports neural cell growth. J Neurosci Res 87, 3198-3206.
23. Aizman, I., Tirumalashetty, B. J., McGrogan, M., Case, C. C., 2014. Comparison of the neuropoietic activity of gene-modified versus parental mesenchymal stromal cells and the identification of soluble and extracellular matrix-related neuropoietic mediators. Stem Cell Res Ther 5, 29.
24. Mattson, M. P., 2008. Glutamate and neurotrophic factors in neuronal plasticity and disease. Ann N Y Acad Sci 1144, 97-112.
25. Hamanaka, R. B., O′Leary, E. M., Witt, L. J., Tian, Y., Gökalp, G. A., Meliton, A. Y., . . . & Mutlu, G. M., 2019. Glutamine metabolism is required for collagen protein synthesis in lung fibroblasts. American journal of respiratory cell and molecular biology, 61(5), 597-606.
26. Romanos, J., Benke, D., Saab, A. S., Zeilhofer, H. U., Santello, M., 2019. Differences in glutamate uptake between cortical regions impact neuronal NMDA receptor activation. Commun Biol 2, 127.
27. Heeger, D. J., & Ress, D., 2002. What does fMRI tell us about neuronal activity?. Nature reviews neuroscience, 3(2), 142-151.

We claim:

1. A method of inducing tonic release of glutamate in a subject, the method comprising:
administering vandefitemcel to a region of the brain of the subject, wherein the vandefitemcel are cells descended from mesenchymal stem cells (MSCs) transiently-transfected by a polynucleotide encoding a Notch intracellular domain (NICD).

2. The method of claim 1, wherein administering the vandefitemcel further comprises administering the vandefitemcel by intracerebral implantation.

3. The method of claim 2, wherein the vandefitemcel tonically release glutamate to neurons of the brain when implanted via intracerebral implantation.

4. The method of claim 2, wherein administering the vandefitemcel further comprises injecting the vandefitemcel at multiple sites within the region of the brain.

5. The method of claim 2, wherein administering the vandefitemcel further comprises administering the vandefitemcel stereotactically via a burr hole in the skull of the subject.

6. The method of claim 1, wherein the region of the brain is a forebrain of the subject.

7. The method of claim 1, wherein the region of the brain is a site of injury or disease.

8. The method of claim 1, wherein the region of the brain is a hippocampus of the subject.

9. The method of claim 1, wherein administering the vandefitemcel further comprises administering the vandefitemcel by parenteral administration.

10. The method of claim 1, wherein the vandefitemcel is suspended in a sterile isotonic crystalloid solution.

11. The method of claim 1, wherein administering the vandefitemcel further comprises administering between about 1.0 million cells and 10.0 million cells.

12. The method of claim 1, wherein the vandefitemcel is made by a method comprising:
providing a culture of MSCs;
contacting the culture of MSCs with the polynucleotide encoding the NICD, wherein the polynucleotide does not encode a full-length Notch protein,
selecting cells that comprise the polynucleotide; and
further culturing the selected cells in the absence of selection for the polynucleotide.

13. The method of claim 1, wherein the MSCs are human bone marrow-derived cells.

14. The method of claim 1, further comprising:
capturing functional magnetic resonance imaging (fMRI) scans of regions of the brain of the subject;
selecting one region of the brain showing neuronal activity based on the fMRI scans; and
administering the vandefitemcel to the one region of the brain.

15. A method of increasing synapse formation in neurons, the method comprising:
administering vandefitemcel to a region of the brain of the subject comprising the neurons, wherein the vandefitemcel are cells descended from mesenchymal stem cells (MSCs) transiently-transfected by a polynucleotide encoding a Notch intracellular domain (NICD).

16. The method of claim 15, wherein administering the vandefitemcel further comprises administering the vandefitemcel by intracerebral implantation.

17. The method of claim 16, wherein the vandefitemcel tonically release glutamate to the neurons when implanted via intracerebral implantation.

18. The method of claim 16, wherein administering the vandefitemcel further comprises injecting the vandefitemcel at multiple sites within the region of the brain.

19. The method of claim 16, wherein administering the vandefitemcel further comprises administering the vandefitemcel stereotactically via a burr hole in the skull of the subject.

20. The method of claim 15, wherein the region of the brain is a forebrain of the subject.

21. The method of claim 15, wherein the region of the brain is a site of injury or disease.

22. The method of claim 16, wherein the region of the brain is a hippocampus of the subject.

23. The method of claim 15, wherein administering the vandefitemcel further comprises administering the vandefitemcel by parenteral administration.

24. The method of claim 15, wherein the vandefitemcel is suspended in a sterile isotonic crystalloid solution.

25. The method of claim 15, wherein administering the vandefitemcel further comprises administering between about 1.0 million cells and 10.0 million cells.

26. The method of claim 15, wherein the vandefitemcel is made by a method comprising:
providing a culture of mesenchymal stem cells;
contacting the culture of mesenchymal stem cells with the polynucleotide encoding the NICD, wherein the polynucleotide does not encode a full-length Notch protein,
selecting cells that comprise the polynucleotide; and
further culturing the selected cells in the absence of selection for the polynucleotide.

27. The method of claim 15, wherein the MSCs are human bone marrow-derived cells.

28. The method of claim 15, further comprising:
capturing functional magnetic resonance imaging (fMRI) scans of regions of the brain of the subject;
selecting one region of the brain showing neuronal activity based on the fMRI scans; and
administering the vandefitemcel to the one region of the brain.

29. A composition for increasing synapse formation in neurons, the composition comprising:
vandefitemcel in an amount between about 1.0 million cells and 10.0 million cells, wherein the vandefitemcel is produced by modifying mesenchymal stem cells derived from bone marrow; and
one or more pharmaceutically acceptable excipients.

30. The composition of claim 29, wherein the vandefitemcel is made by a process comprising:
providing a culture of mesenchymal stem cells;
contacting the culture of mesenchymal stem cells with a polynucleotide encoding a Notch intracellular domain (NICD), wherein the polynucleotide does not encode a full-length Notch protein,
selecting cells that comprise the polynucleotide; and
further culturing the selected cells in the absence of selection for the polynucleotide.

31. The composition of claim 30, wherein the mesenchymal stem cells are human bone marrow-derived cells.

32. The composition of claim 29, wherein the mesenchymal stem cells are transiently-transfected with a plasmid vector comprising the polynucleotide encoding the NICD.

33. The composition of claim 29, wherein the one or more pharmaceutically acceptable excipients comprises at least one of buffers, proteins, stabilizers, and preservatives.

34. The composition of claim 29, wherein the vandefitemcel is suspended in a sterile isotonic crystalloid solution.

35. The composition of claim 29, wherein the one or more pharmaceutically acceptable excipients comprises carriers or diluents.

36. A method of inducing release of glutamate in a subject, the method comprising:
capturing functional magnetic resonance imaging (fMRI) scans of regions of the brain of the subject;
selecting a region of the brain showing neuronal activity based on the fMRI scans; and
administering vandefitemcel to the region of the brain selected, wherein the vandefitemcel are cells descended from mesenchymal stem cells (MSCs) transiently-transfected by a polynucleotide encoding a Notch intracellular domain (NICD), and wherein the vandefitemcel release glutamate after being administered via intracerebral implantation.

37. A method of increasing synapse formation in neurons, the method comprising:
capturing functional magnetic resonance imaging (fMRI) scans of regions of the brain of the subject;
selecting a region of the brain showing neuronal activity based on the fMRI scans; and
administering vandefitemcel to the region of the brain selected comprising the neurons, wherein the vandefitemcel are cells descended from mesenchymal stem cells (MSCs) transiently-transfected by a polynucleotide encoding a Notch intracellular domain (NICD), and wherein the vandefitemcel release glutamate to the neurons after being administered via intracerebral implantation.
